(19) United States
(12) Patent Application Publication
(10) Pub. No.: US 2023/0298691 A1
(43) Pub. Date: Sep. 21, 2023

(54) DISTRIBUTED GENETIC TESTING SYSTEMS UTILIZING SECURE GATEWAY SYSTEMS AND NEXT-GENERATION SEQUENCING ASSAYS

(71) Applicant: Aspira Women's Health, Trumbull, CT (US)

(72) Inventors: Lesley Northrup, New York, NY (US); Thomas Greco, Monroe, CT (US); Nitin Bhardwaj, Jersey City, NJ (US); Justin DeGrazia, Norwalk, CT (US); Pierre Davidoff, Brooklyn, NY (US)

(21) Appl. No.: 17/652,581

(22) Filed: Feb. 25, 2022

(51) Int. Cl. G16B 20/20; C12N 15/10; C12Q 1/6883; G16B 40/20

(52) U.S. Cl. CPC G16B 20/20; C12N 15/1065; C12Q 1/6883; G16B 40/20; C12Q 1/6869

(57) ABSTRACT

Various embodiments of the present invention introduce techniques for performing genetic screening using a cloud-based genetic testing framework. In some embodiments, a genetic testing server uses a set of oligonucleotide probes for detecting targeted genes based on sample data objects with an oligonucleotide or primer set. To overcome the challenges associated with variability of output data across client devices (e.g., across laboratories) which is a major roadblock to implementing a cloud-based genetic testing framework, various embodiments introduce techniques for validating assays’ with strong baseline metrics to ensure the identification of “user” error vs “assay performance” error, which increases transferability across clients. Moreover, in embodiments, an assay that combines the reagent components with the patient’s genomic DNA (gDNA) in a single tube process, limiting transfer steps and reducing outside contamination is provided.

### SEQUENCE LISTING

The instant application contains a Sequence Listing which has been submitted electronically in ASCII format and is hereby incorporated by reference in its entirety. Said ASCII copy, created on Feb. 24, 2022, is named 548932seqlisting.TXT and is 13,479,242 bytes in size.

### BACKGROUND

Genetics is the make-up of who we are as a human species and affects all individuals, regardless of race or ethnicity. Followed by rapid advancements in technology and mapping of the human genome, genetic testing has come to the forefront of clinical management, allowing for the study of human diseases at a fraction of the cost. A new clinical management paradigm exists today, when identifying risk of disease almost always warrants some type of genetic testing.

Key statistics highlight the importance of implementing genetic screening/testing across standard healthcare clinical management. Seven percent of the general population has a rare genetic condition, many of which are undiagnosed. It has been reported that in 7.9% of patients studied, a pathogenic or likely pathogenic (P/LP) variant was identified, which would have been missed when following the current National Comprehensive Cancer Network (NCCN) guidelines for breast/ovarian cancer testing. Ninety percent of the general population are carriers of an inherited disease. Sixteen percent of individuals carry a moderate risk variant, which may change clinical care. Studies have shown 80% of individuals have been identified with a genetic variant associated with a known pharmacologic response to a drug dose response.

However, despite these advances in genetic technology, genetic testing remains largely fragmented and expensive. The market for high throughput germline genetic testing is one of the largest growth sectors of laboratory testing in the healthcare industry. Prenatal tests including non-invasive prenatal testing (NIPT) and carrier screening account for the highest percentage of spend over the last 10 years ranging from 33% to 43% of the genetic testing market, followed by hereditary cancer tests at approximately 30%.

Currently, supporting the internalization of genetic tests requires high investment both from staffing personnel as well as building a complex infrastructure to analyze genetic data at scale. Current solutions involve multiple workflows that lead to increased time, complexity, and overall significant cost. Thus, many labs cannot implement and support multiple or advanced genetic testing due to the high cost involved in building it. This puts barriers in place, optically viewed as daunting with a big capital investment. Therefore, most clinics and/or clinical labs choose to outsource. There remains a need to democratize genetic testing and allow any healthcare provider the capabilities to offer patients an affordable clinical genetic testing option.

Through applied effort, ingenuity, and innovation, many of these identified problems have been solved by developing solutions that are included in embodiments of the present subject matter, many examples of which are described in detail herein.

### BRIEF SUMMARY

An embodiment of the invention is a computer-implemented method for generating a report data structure for a genetic testing request that is received from an integrated client device, the computer-implemented method comprising:
contacting a sample from a subject with an oligonucleotide or primer set, said set comprising at least one oligonucleotide probe or primer pair, wherein the at least one oligonucleotide probe or primer pair is labelled and configured to bind to at least one nucleic acid sequence in the sample;
amplifying the at least one nucleic acid sequence in the sample so as to generate at least one amplification product;
sequencing the at least one amplification product using one or more next generation sequencing operations to generate library preparation product sequencing data;
transmitting the library preparation product sequencing data from the integrated client device to a genetic testing server;
identifying, based on the library preparation product sequencing data, a sequence data structure and a client identifier for the integrated client device;
storing the sequence data structure on an encrypted storage framework and in association with the client identifier;
extracting, from the sequence data structure, a) a raw sequence data object, and b) a sample data object;
generating a sample data structure comprising the raw sequence data object, and the sample data object;
generating the report data structure based on the sample data structure; and
transmitting the report data structure from the genetic testing server to the integrated client device.

Another embodiment is a kit, comprising
i) at least one oligonucleotide probe or primer pair, wherein each oligonucleotide probe or primer pair is labelled and configured to amplify in an amplification reaction at least one nucleic acid sequence in a sample; and
ii) an apparatus configured to programmatically enable the analysis of amplification product sequencing data, the apparatus comprising at least a processor, and a memory associated with the processor having computer coded instructions therein, with the computer coded instructions configured to, when executed by the processor, cause the apparatus to:
   a. receive, from an integrated client device, amplification product sequencing data;
   b. identify, based on the amplification product sequencing data, a sequence data structure and a client identifier for the integrated client device;
   c. store the sequence data structure on an encrypted storage framework and in association with the client identifier;
   d. extract, from the sequence data structure, a) a carrier testing raw sequence data object or a cancer testing raw sequence data object, and b) a sample data object;
   e. generate a sample data structure comprising the carrier testing raw sequence data object or the cancer testing raw sequence data object, and the sample data object;
   f. generate the report data structure based on the sample data structure; and
   g. transmit the report data structure to the integrated client device.

Some embodiments are directed to methods, systems, apparatuses, and computer program products for an apparatus configured to enable the analysis of genetic testing raw sequence data via an electronic platform. The apparatus comprises a processor, and a memory associated with the processor having computer coded instructions therein, with the computer coded instructions configured to, when executed by the processor, cause the apparatus to enable the analysis of genetic testing raw sequence data.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates an exemplary architecture for a distributed genetic testing system, according to embodiments of the present disclosure.

FIG. 2 illustrates an exemplary secure gateway device for use with embodiments disclosed herein.

FIG. 3 illustrates a flowchart illustrating exemplary operations of a secure gateway device for use with embodiments of the present disclosure.

FIG. 4 is an exemplary schematic diagram illustrating the encrypted storage framework 113 where each client will have their own dedicated, HIPAA-compliant, encrypted cloud-based file, completely protected from other client/patient data with the ASG portal infrastructure.

FIG. 5 is an exemplary schematic diagram illustrating that ASG enables the service lab to run the entire test internally and not rely on “outsourced” labs, and therefore incur revenue a reduce financial leakage due to high-fees incurred by outsourcing. Standard ASG framework: Clients have a molecular license and next generation sequencing already in-house with workflows, full time employees (FTEs), space, overhead. Clients have complete control of the entire Laboratory Developed Test (LDT) process from blood draw and wet lab to clinical report.

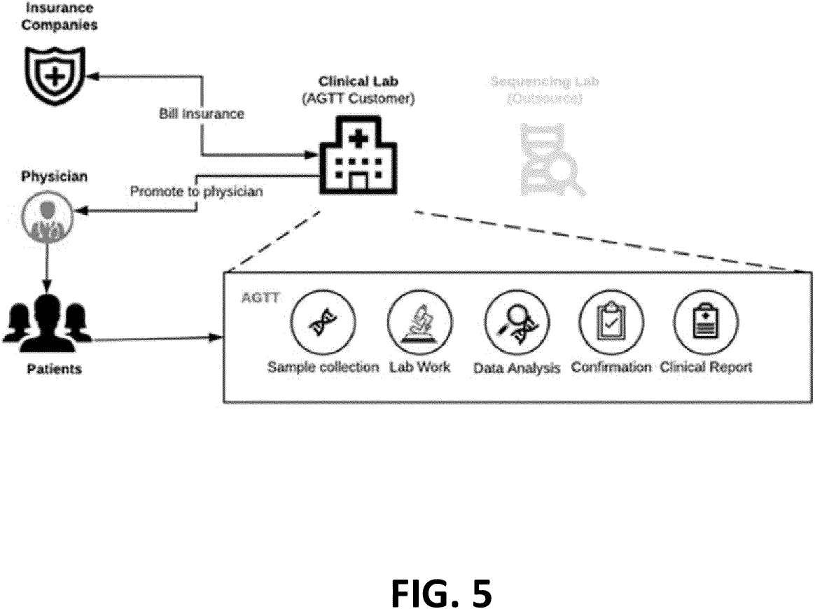

FIG. 6 is an exemplary schematic diagram outlining the variant analysis tool. The input for the process is a raw sequencing data file, i.e. FASTQ files. Raw sequencing data files are aligned and then variants are called (secondary analysis) using benchmarked algorithms. The platform provides the tertiary analysis in which variants are automatically being interpreted and characterized based on disease prevalence. The interpreted variants are part of the final clinical report which is generated using the platform. The platform also provides the entire workflow management as quality metrics on the sequencing data to provide to the laboratory as part of the Quality Assurance metrics required by laboratory regulatory agencies CLIA and CAP.

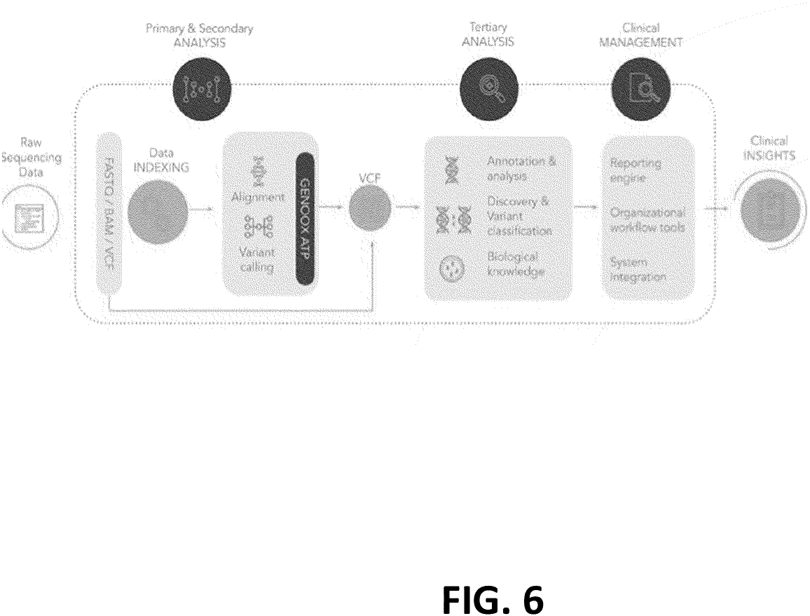

FIG. 7 is an exemplary schematic diagram showing the process of how a sample is received and integrated into the Bioinformatics pipeline. Once the sample has been collected it is managed in the service labs laboratory information system (LIS) and run using ASG chemistry, then the sample is processed and sequenced. Raw sequencing data automatically uploads to the ASG ‘frontend portal’ in combination with the patient’s medical information as collected from the sample requisition form. The raw sequencing data is analyzed using the bioinformatics pipeline to perform variant calling. Called variants are automatically interpreted using the interpretation engine. A preanalytical assessment of the finalized variant interpretation is reviewed by a clinical board molecular geneticist and finalized into a ‘pre-signed’ patients report. The ‘pre-signed’ patients report is then delivered to the service lab through the frontend portal and pulled back into the service labs LIS. The service lab director confirms the results and signs the clinical patient report.

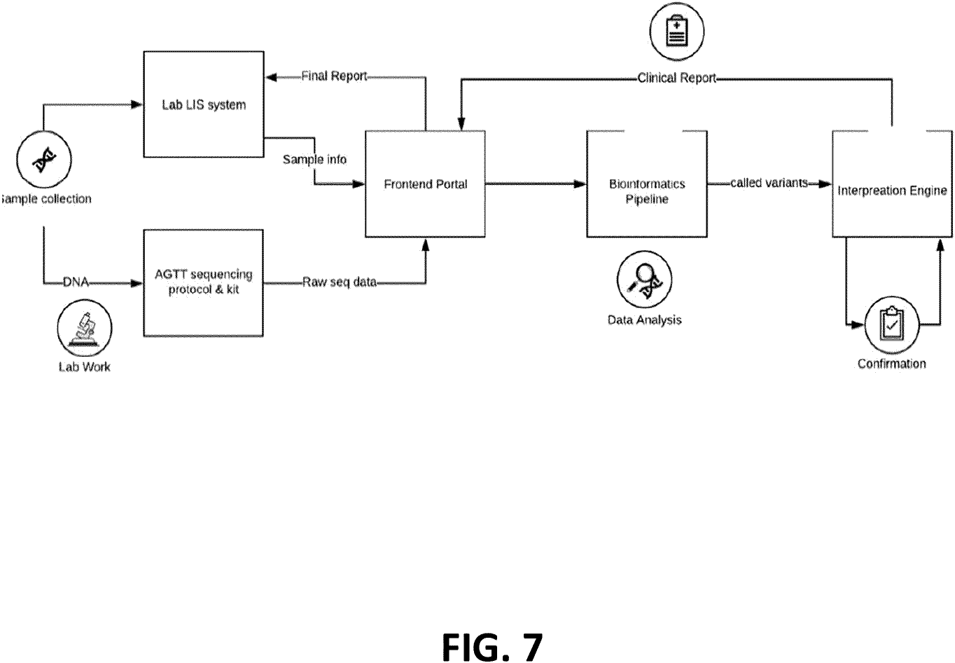

FIG. 8 is an exemplary schematic diagram that illustrates the NIPT wet-bench workflow as compared to the ASG chemistry protocol. Each step overlaps, and three different types of assays can be run on the same instrumentation.

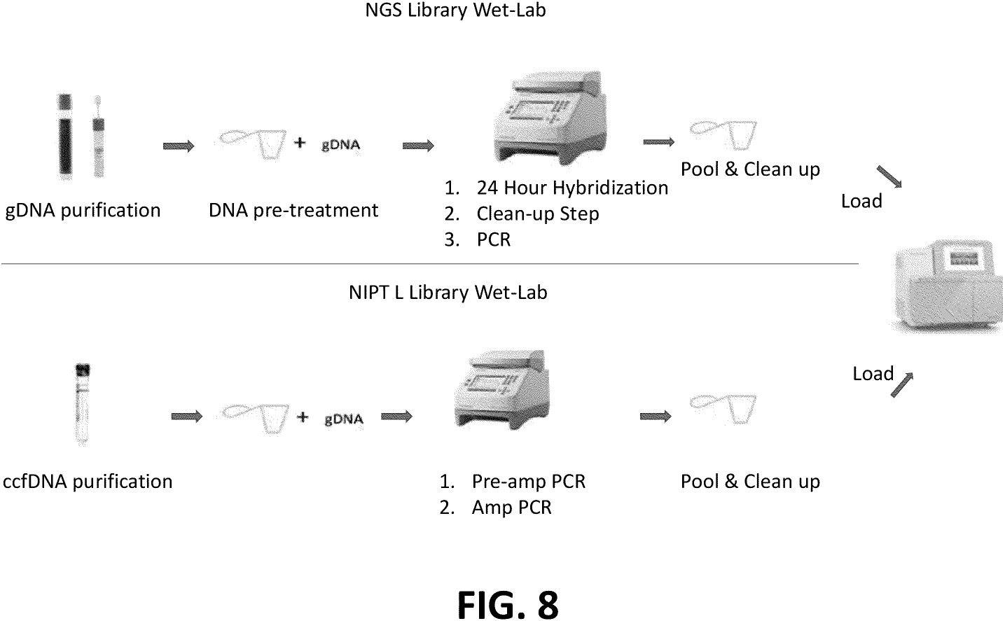

### DETAILED DESCRIPTION

In embodiments, the subject matter described herein is an all-in-one, end-to-end screening system that provides for testing materials, cloud-based analysis, and reporting of genetic variations, at a relatively low cost to the institutions performing the screening. It allows for institutions that may lack the resources traditionally required for genetic testing services to offer these services, using an updateable cloud-based system with relatively little infrastructural investment, with common sequencing equipment that may already be present in their laboratories. The combination of testing materials (such as oligonucleotide probes), cloud-based bioinformatics analysis, and reporting is not currently offered. The systems described herein provide an easy and economical means for patients, health-care providers, and researchers alike to receive vital information regarding genetic variants.

Provided herein is an assay for genetic screening. In an embodiment, the genetic screening is carrier or hereditary cancer screening. The first potential challenge in developing this technology was around the chemistry design and its ability to achieve the necessary accuracy and precision in a given assay. Currently, there are gene regions that are “low covered” with the existing chemistry, which forces laboratories to use a secondary technology to ensure precision coverage of the genes of interest. Similarly, today due to problematic regions in the genome, e.g., copy number variants (CNVs), pseudogenes, current bioinformatic pipelines are inadequate. Described herein are a set of oligonucleotide probes for detecting variants in such regions of interest. In embodiments, provided herein is a set of oligonucleotide probes for detecting targeted genes comprising at least the genes listed in Table 3.

Additionally, each laboratory’s test assay performs differently across different end users and there will inevitably be some variability of the output data. Therefore, to ensure that the platform described herein can account for these variables, the assays’ performance was validated with strong baseline metrics to ensure the identification of “user” error vs “assay performance” error, which increases transferability across labs. Furthermore, the overall wet-lab design of the test assay, which involves a simple workflow with less hands-on technologist time, minimizes error rate.

Described herein are assays that offer carrier or hereditary cancer screening. In embodiments, a Technology Transfer to implement a Laboratory Developed Test (LDT) is provided. In another embodiment, a previously validated, next generation sequencing (NGS) assay is provided. In one embodiment, a wet lab kit for carrier and/or hereditary cancer screening is provided.

In embodiments, an assay that combines the reagent components with the patient’s genomic DNA (gDNA) in a single tube process, limiting transfer steps and reducing outside contamination is provided. In embodiments, an assay where the entire wet-lab bench work will be reduced to, for example, about 90-min of hands-on time due to the design of the chemistry and would not require multiple purification steps as observed in other chemistries which increases the potential for laboratory error that may lead to inaccurate results. In embodiments, the majority of the assay runtime are hands-off processes. In embodiments, the assay runtime comprises a 4-24-hour hybridization and a 24-hour run processing time on a sequencing instrument. In embodiments, the 24-hour hybridization allows binding of the gDNA to the synthetic oligonucleotides that targets the genomic regions of interest. The simplified chemistry workflow utilized herein allows laboratories that do not have previous experience in molecular biology techniques to implement a workflow seamlessly with little-to-no overhead with a low-learning curve and limited troubleshooting.

In embodiments, the assay provided herein can be used in conjunction with ASPIRA Synergy Genetics technology transfer (ASG). ASG is a fully automated and customizable genetic testing solution which greatly simplifies workflows by taking well-established high throughput bioinformatics technologies backed by artificial intelligence, thereby simplifying it for clinical laboratories, regardless of operational size and footprint, without compromising on the quality of the test.

Currently, genetic tests for hereditary cancer and carrier screening are run by large specialty organizations, esoteric laboratories, regional laboratories, and direct to consumer providers. While the market represents an opportunity, there are technical hurdles. Erstwhile, the market has been restricted to few companies that possess the intricate knowledge, technology, and personnel to run such testing as hospital and healthcare organizations. However, operational, clinical, and analytic challenges remain unsolved and preclude launch of a competitive product. The reasons for this are multifaceted, including: complexity, e.g., types of panels, number of genes offered, variants of interest, wet lab, reagents, personnel, curation, keeping up with clinical guidelines, variant reclassification, and workflows, e.g., next generation sequencing for inherited cancer and carrier screening generally requires multiple workflows (up to 6) to capture all of the variants of interest with the highest sensitivity in complex genes and regions, and may require confirmation by a secondary technology method if covered at low sequencing coverage. The methods, kits, and compositions for genetic testing described herein have the potential to substantially increase access to these much needed tests.

Various embodiments of the inventions now will be described more fully hereinafter, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level.

### Exemplary Definitions

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “oligonucleotide probe” or “probe” refers to a single-stranded nucleotide sequence that is complementary to a region of interest. In some embodiments, the probe has a dye or other detectable label attached thereto.

The term “primer” refers to a single-stranded nucleotide sequence that is complementary to a region of interest that is to be amplified.

The term “bind,” when used in relation to nucleic acid sequences, may refer to any way in which two complementary nucleic acid sequences adhere to each other, including hybridization or annealing.

The term “variant” refers to an amino acid or nucleic acid sequence (or an organism or tissue) that is different from the majority of the population but is still sufficiently similar to the common mode to be considered to be one of them (e.g., splice variants).

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ± 0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p ≤0.05.

The term “sequence data object” may refer to a data construct that is configured to describe a message from an integrated client device comprising raw sequence data. In embodiments, the raw sequence data are FASTQ files. In embodiments, the sequence data object is a raw sequence data object. In embodiments, the raw sequence data object comprises a carrier testing raw sequence data object or a cancer testing raw data object. In embodiments, the sequence data object further comprises a sample data object. In embodiments, the sequence data object is received from an integrated client device such as a next generation sequencing (NGS) device. In embodiments, the NGS device is an Illumina sequencer.

The term “sample data object” may refer to a data construct that is configured to describe a message from an integrated client device that includes sample identification information or patient information. In embodiments, the sample data object is received from a second integrated client device such as a laboratory information system (LIS). In some embodiments, the sample data object comprises one or more arrays, where each array value describes a genetic feature value associated with the patient.

“Sequence identity” or “identity” in the context of two polynucleotides or polypeptide sequences refers to to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “external resource” may refer to a combination of one or more computing devices that is configured to execute a software program, application, platform, or service that is configured to communicate with the technology transfer system. In some embodiments, the external resource may communicate with the ASG system, and vice versa, through one or more application program interfaces (APIs). In some embodiments, the external resource receives tokens or other authentication credentials that are used to facilitate secure communication between the genetic testing server and the ASG system in view of ASG system network security layers or protocols (e.g., network firewall protocols). In embodiments, the one or more external resources 101 communicate with the secure gateway computing device 103 and vice versa via a communication network 105. In embodiments, the bioinformatics pipeline and interpretations engine are components of computing device 103. In embodiments, the secure gateway computing device 103 comprises the bioinformatics pipeline and interpretations engine.

The term “library preparation product sequencing data” may refer to the output of performing either ligation-based library preparation operations or tagmentation-based library preparation operations with respect to an amplification product. In some embodiments, performing a library preparation operation comprises fragmenting and end-repairing DNA or RNA samples of the amplification product.

The term “client identifier” may refer to a data construct that uniquely identifies a client device and/or a client entity. In some embodiments, the client identifier is used to store a sequence data structure associated with the corresponding client device and/or the corresponding client entity.

The term “sample data structure” may refer to a data construct that comprises a raw sequence data object and a sample data object. In some embodiments, the sample data structure is used to generate a report data structure, and the report data structure may then be transmitted to a client device.

### Exemplary System Architectures

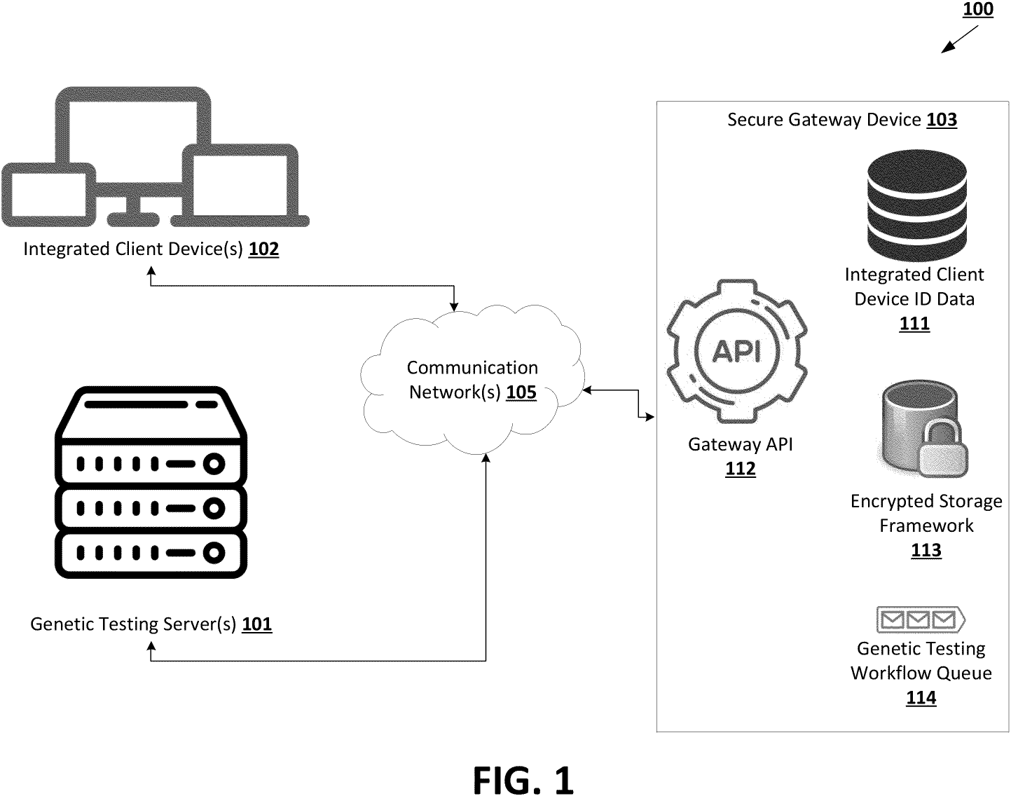

FIG. 1 is a block diagram of an exemplary architecture for a distributed genetic testing system 100 that can be used to enable access to the genetic testing functionalities and genetic machine learning functionalities described herein by integrated client devices 102. As depicted in FIG. 1, the distributed genetic testing system 100 comprises one or more external resources 101, one or more integrated client devices 102, and a secure gateway device 103 that can communicate with each other via one or more communication networks 105.

As depicted in FIG. 1, the distributed genetic testing system 100 comprises a set of integrated client devices 102, where each integrated client device 102 may have previously been registered with a secure gateway computing device 103 by associating identifying data associated with the integrated client device 102 with a unique client identifier of a client profile that is associated with the integrated client device 102.

In some embodiments, an integrated client device 102 may be configured to provide a genetic testing request comprising one or more sequence data structures to a frontend portal 112 (e.g., a gateway device application programming interface (API)) of the secure gateway computing device 103. In response to the genetic testing request, the frontend portal 112 may be configured to store one or more sequence data structures associated with genetic testing request on the encrypted storage framework 113 which is an enhanced security storage framework and as part of a client file repository for the client identifier; and generate a genetic testing workflow for the genetic testing request in the genetic testing workflow queue 114.

During a defined time window (e.g., periodically), a bioinformatics pipeline 114 of the secure gateway computing device 103 may be configured to call an interpretation engine 115 to perform one or more genetic testing operations and/or one or more genetic machine learning operations using the external resources 101. Via performing the noted operations, the interpretation engine 115 may be configured to generate a clinical report that may then be provided as output data to a requesting integrated client devices 102.

### Exemplary Secure Gateway Devices

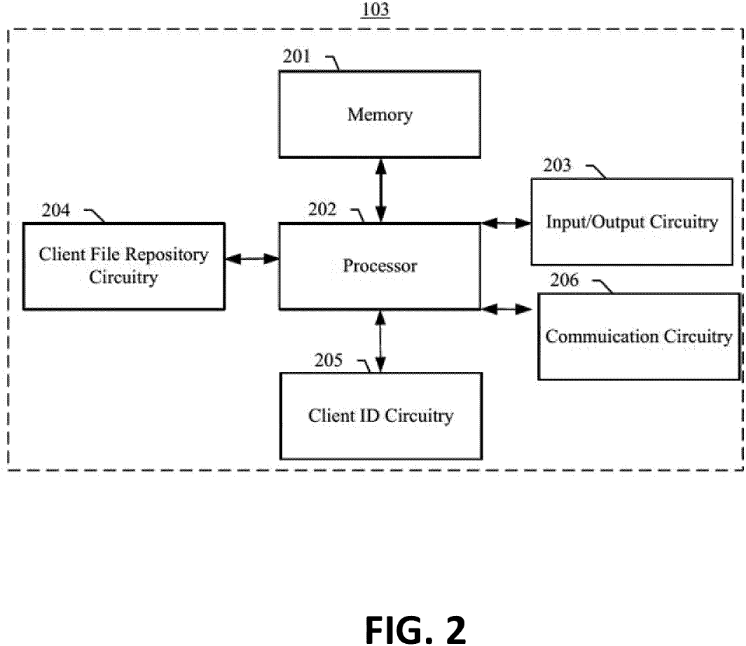

An exemplary architecture for a secure gateway device 103 is depicted in FIG. 2. As illustrated in FIG. 2, the exemplary apparatus 103 may comprise processor 202, memory 201, input-output circuitry 203, communications circuitry 206, client file repository circuitry 204, and client ID circuitry 205. The apparatus 103 may be configured to execute the operations described herein. Although some of these components 201-206 are described with respect to their functional capabilities, it should be understood that the particular implementations necessarily include the use of particular hardware to implement such functional capabilities. It should also be understood that certain of these components 201-206 may include similar or common hardware. For example, two sets of circuitry may both leverage use of the same processor, network interface, storage medium, or the like to perform their associated functions, such that duplicate hardware is not required for each set of circuitry.

The use of the term “circuitry” as used herein with respect to components of the apparatus 103 therefore includes particular hardware configured to perform the functions associated with respective circuitry described herein. Of course, while the term “circuitry” should be understood broadly to include hardware, in some embodiments, circuitry may also include software for configuring the hardware. For example, in some embodiments, “circuitry” may include processing circuitry, storage media, network interfaces, input-output devices, and other components. In some embodiments, other elements of the apparatus 103 may provide or supplement the functionality of particular circuitry. For example, the processing circuitry 202 may provide processing functionality, memory 201 may provide storage functionality, and communications circuitry 206 may provide network interface functionality, among other features.

In some embodiments, the processor 202 (and/or co-processor or any other processing circuitry assisting or otherwise associated with the processor) may be in communication with the memory 201 via a bus for passing information among components of the apparatus. The memory 201 may be non-transitory and may include, for example, one or more volatile and/or nonvolatile memories. For example, the memory 201 may be an electronic storage device (e.g., a computer readable storage medium). In another example, the memory 201 may be a non-transitory computer-readable storage medium storing computer-executable program code instructions that, when executed by a computing system, cause the computing system to perform the various operations described herein. The memory 201 may be configured to store information, data, content, signals applications, instructions (e.g., computer-executable program code instructions), or the like, for enabling the apparatus 103 to carry out various functions in accordance with example embodiments of the present disclosure. It will be understood that the memory 201 may be configured to store partially or wholly any electronic information, data, data structures, embodiments, examples, figures, processes, operations, techniques, algorithms, instructions, systems, apparatuses, methods, or computer program products described herein, or any combination thereof.

The processor 202 may be embodied in a number of different ways and may, for example, include one or more processing devices configured to perform independently. Additionally or alternatively, the processor 202 may include one or more processors configured in tandem via a bus to enable independent execution of instructions, pipelining, multithreading, or a combination thereof. The use of the term “processor” may be understood to include a single core processor, a multi-core processor, multiple processors internal to the apparatus, remote or “cloud” processors, or a combination thereof.

In an exemplary embodiment, the processor circuitry 202 may be configured to execute instructions stored in the memory 201 or otherwise accessible to the processor 202. Alternatively or additionally, the processor 202 may be configured to execute hard-coded functionality. As such, whether configured by hardware or software methods, or by a combination of hardware with software, the processor 202 may represent an entity (e.g., physically embodied in circuitry) capable of performing operations according to an embodiment of the present disclosure while configured accordingly. As another example, when the processor 202 is embodied as an executor of program code instructions, the instructions may specifically configure the processor to perform the operations described herein when the instructions are executed.

In some embodiments, the apparatus 103 may include input-output circuitry 203 that may, in turn, be in communication with processor 202 to provide output to the user and, in some embodiments, to receive input such as a command provided by the user. The input-output circuitry 203 may comprise a user interface, such as a graphical user interface (GUI), and may include a display that may include a web user interface, a GUI application, a mobile application, an integrated client device, or any other suitable hardware or software. In some embodiments, the input-output circuitry 203 may also include a keyboard, a mouse, a joystick, a display device, a display screen, a touch screen, touch areas, soft keys, a microphone, a speaker, or other input-output mechanisms. The processor 202, input-output circuitry 203 (which may utilize the processor 202), or both may be configured to control one or more functions of one or more user interface elements through computer-executable program code instructions (e.g., software, firmware) stored in a non-transitory computer-readable storage medium (e.g., memory 201). Input-output circuitry 203 is optional and, in some embodiments, the apparatus 110 may not include input-output circuitry. For example, where the apparatus 103 does not interact directly with the user, the apparatus 103 may generate user interface data for display by one or more other devices with which one or more users directly interact and transmit the generated user interface data to one or more of those devices.

The communications circuitry 206 may be any device or circuitry embodied in either hardware or a combination of hardware and software that is configured to receive or transmit data from or to a network or any other device, circuitry, or module in communication with the apparatus 103. In this regard, the communications circuitry 206 may include, for example, a network interface for enabling communications with a wired or wireless communication network. For example, the communications circuitry 206 may include one or more network interface cards, antennae, buses, switches, routers, modems, and supporting hardware and/or software, or any other device suitable for enabling communications via a network. In some embodiments, the communication interface may include the circuitry for interacting with the antenna(s) to cause transmission of signals via the antenna(s) or to handle receipt of signals received via the antenna(s). These signals may be transmitted or received by the apparatus 103 using any of a number of Internet, Ethernet, cellular, satellite, or wireless technologies, such as IEEE 802.11, Code Division Multiple Access (CDMA), Global System for Mobiles (GSM), Universal Mobile Telecommunications System (UMTS), Long-Term Evolution (LTE), Bluetooth® v1.0 through v5.0, Bluetooth Low Energy (BLE), infrared wireless (e.g., IrDA), ultra-wideband (UWB), induction wireless transmission, Wi-Fi, near field communications (NFC), Worldwide Interoperability for Microwave Access (WiMAX), radio frequency (RF), RFID, or any other suitable technologies.

The client file repository circuitry 204 includes hardware components designed or configured to receive, process, generate, and transmit data, such as the sample data structure, raw sequence data object, and report data structure. In some embodiments, the client file repository circuitry 204 may be in communication with the communications circuitry 206 and thus configured to receive data from the communications circuitry 206. As described above and as will be appreciated based on this disclosure, embodiments of the present disclosure may be configured as systems, apparatuses, methods, mobile devices, backend network devices, computer program products, other suitable devices, and combinations thereof. Accordingly, embodiments may comprise various means including entirely of hardware or any combination of software with hardware. Furthermore, embodiments may take the form of a computer program product on at least one non-transitory computer-readable storage medium having computer-readable program instructions (e.g., computer software) embodied in the storage medium. Any suitable computer-readable storage medium may be utilized including non-transitory hard disks, CD-ROMs, flash memory, optical storage devices, or magnetic storage devices. As will be appreciated, any computer program instructions and/or other type of code described herein may be loaded onto a computer, processor or other programmable apparatus’s circuitry to produce a machine, such that the computer, processor, or other programmable circuitry that executes the code on the machine creates the means for implementing various functions, including those described herein.

The client ID circuitry 205 includes hardware components designed or configured to receive, process, generate, and transmit data, such as the sample data structure, raw sequence data object, and report data structure. In some embodiments, the client ID circuitry 205 may be in communication with the communications circuitry 206 and thus configured to receive data from the communications circuitry 206. As described above and as will be appreciated based on this disclosure, embodiments of the present disclosure may be configured as systems, apparatuses, methods, mobile devices, backend network devices, computer program products, other suitable devices, and combinations thereof. Accordingly, embodiments may comprise various means including entirely of hardware or any combination of software with hardware. Furthermore, embodiments may take the form of a computer program product on at least one non-transitory computer-readable storage medium having computer-readable program instructions (e.g., computer software) embodied in the storage medium. Any suitable computer-readable storage medium may be utilized including non-transitory hard disks, CD-ROMs, flash memory, optical storage devices, or magnetic storage devices. As will be appreciated, any computer program instructions and/or other type of code described herein may be loaded onto a computer, processor or other programmable apparatus’s circuitry to produce a machine, such that the computer, processor, or other programmable circuitry that executes the code on the machine creates the means for implementing various functions, including those described herein.

### Exemplary Distributed Genetic Testing Techniques

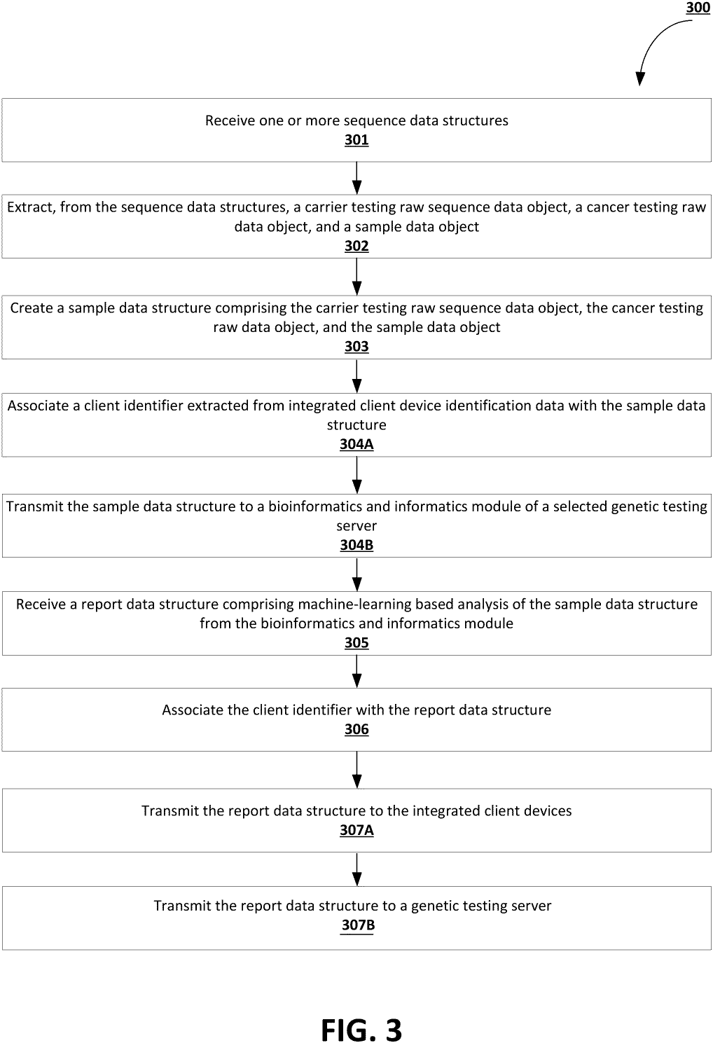

Referring to FIG. 3, an example data flow 300 attributable to enabling the analysis of genetic testing raw sequence data is provided. The operations described in connection with FIG. 3 may, for example, be performed by one or more components described with reference to apparatus 103 shown in FIG. 2 (e.g., by or through the use of one or more of processor 202, memory 201, input-output circuitry 203, communications circuitry 206, client file repository circuitry 204, client ID circuitry 205, any other suitable circuitry, and any combination thereof); by any other component described herein; or by any combination thereof. Various operations of the data flow 300 may in some embodiments be performed by the bioinformatics pipeline 114 and the interpretation engine 115.

In exemplary data flow 300, secure gateway computing device 103 receives at block 301 from one or more integrated client devices 102 via a communications network 105, one or more sequence data structures. In embodiments, the one or more sequence data structures are associated with a client identifier. In embodiments, associating the client identifier with the same data structure results in the storage of the sample data structure in the encrypted storage framework 113. In embodiments, each client identifier corresponds to a compartmentalized storage area with the encrypted storage framework 113.

In embodiments, at block 302, secure gateway computing device 103 extracts from the sequence data structures a raw sequence data object and a sample data object.

In embodiments, at block 303, secure gateway computing device 103 creates a sample data structure comprising the raw sequence data object and the sample data object. In embodiments, each data object is associated with data and metadata. In embodiments, the metadata comprises a sample ID. In embodiments, the metadata comprises a sample ID associated with a client identifier. In embodiments, the metadata comprises a sample ID and client identifier. In embodiments, each data object has a plurality of records. In embodiments, the plurality of records comprises data and metadata associated with the data object. In embodiments, the sample data structure comprises structured data. In embodiments, the sample data structure comprises both structured and unstructured data. In embodiments, the sample data structure comprises unstructured data.

In embodiments, at block 304A, secure gateway computing device 103 associates a client identifier with the sample data structure. At block 304B, secure gateway computing device 103 transmits the sample data structure to a bioinformatics and informatics module for machine learning-based analysis of a selected external resource 101. At block 304C, the bioinformatics and informatics module perform the bioinformatics analysis operations and interpretation operations based on the sample data structure to generate a report data structure.

In embodiments, at block 305, secure gateway computing device 103 receives the report data structure from the bioinformatics and interpretation module of the selected external resource 101. In embodiments, the report data structure is based on at least the raw sequence data object in the sample data structure.

In embodiments, at block 306, secure gateway computing device 103 associates the client identifier with the report data structure.

In embodiments, at block 307, secure gateway computing device 103 transmits, to the one or more integrated client devices 102 via the communications network 105, the report data structure.

In embodiments, the processing time of a sample is to be < 4 hours following the uploading of the data. In embodiments, any number of samples can be uploaded simultaneously.

To keep up with the demands of providing a virtual “decentralized” product, it was imperative we built a high-throughput cloud-based solution. ASG is designed as a virtual, cloud-based platform that can be implemented in any clinical laboratory with a molecular license and NGS capabilities.

As the technology in laboratories are becoming more advanced, especially in molecular techniques, third-party vendors are building their instrumentation to be fully automated using end-to-end information solutions and can easily integrate virtually for in/out data pulling. With the advancements in Amazon’s HIPAA-compliant cloud-storage services (AWS) in combination with numerous software advancements primarily used in the ‘smart technology’ industry, we have decided to utilize those same capabilities to build our end-product and allow seamless workflow for the service laboratories. Our market analysis showed that the vast majority of the labs are open to using cloud computing, and are already using it today or are transitioning their entire operation over to an electronic medical data transfer solution.

Allowing a cloud-based infrastructure to connect in/out of the client’s laboratory information system also reduces workflow and connection points. The integration system for the cloud-based portal has various monitoring, notification, alerting and audit trails mechanisms. In embodiments, the cloud-based portal utilizes AWS tools such as cloudwatch, cloudtrail, SNS, and SES for notifications and alerts.

### Exemplary Compositions

In embodiments, a technology transfer solution to implement a Laboratory Developed Test (LDT) is provided. In another embodiment, a previously validated, next generation sequencing (NGS) assay is provided. In another embodiment, the NGS assay is provided for a Laboratory Developed Test (LDT) implementation by laboratories. In one embodiment, a wet lab kit for carrier and hereditary cancer screening is provided. In order to provide an assay with high accuracy and precision at a reduced cost, a dedicated capture kit is designed to facilitate the targeted sequencing. In one embodiment, the assay provided herein is run on an Illumina sequencer (e.g., Illumina NextSeq, Illumina HiSeq, or Illumina NovaSeq) or Thermo Fisher sequencer (e.g., ION Torrent).

In embodiments, a screening assay is provided. In an embodiment, the assay is a cancer or carrier screening assay. In embodiments, each sample is analyzed using a library preparation chemistry kit. In embodiments, provided is a novel targeted capture kit dedicated for the sequencing of the genes of interest. In embodiment, the genes of interest are carrier and/or hereditary cancer genes. In embodiments, the chemistry kit does not require extensive equipment and reagent use. In embodiments, the main instrumentation required is a sequencer, such as an Illumina sequencer.

The simplified assay workflow allows laboratories that do not have previous experience in molecular biology techniques to implement a workflow seamlessly with little-to-no overhead with a low-learning curve and limited troubleshooting. In embodiments, the chemistry is performed with limited steps compared to conventional hereditary cancer or carrier panels. In embodiments, the chemistry is performed in less time than many other standard NGS workflows.

In embodiments, the sequencing kit comprises designed probes for optimal capture of the gene, and regions of interest. In embodiments, the probes are comprised in molecular inversion probes or padlock probes. In embodiments, the probe set screens for at least one of single nucleotide variants (SNV), small insertions / deletions (Indels), copy number variations (CNVs), homologous regions, and pseudogenes. In one embodiment, the probe set screens for 85 hereditary cancer genes. In one embodiment, the probe set screens for 155 carrier genes.

In embodiments, the oligonucleotide probes described herein are comprised in padlock probes (PLPs) or molecular inversion probes (MIPs). Padlock probes (PLPs) are long oligonucleotides, whose ends are complementary to adjacent target sequences. In embodiments, each padlock probe comprises two oligonucleotide sequences connected by a linker sequence. Following hybridization of PLPs/MIPs to the target, gap-filling and ligation result in circularized DNA molecules containing the sequence of the target together for downstream analyses. In embodiments, the oligonucleotide probes have a label attached thereto.

In embodiments, the assay combines the reagent components with the patient’s genomic DNA (gDNA) in a single tube process, limiting transfer steps and reducing outside contamination. In embodiments, the entire wet-lab bench work is significantly reduced compared to other library preparation methods, to about 90-min of hands-on time due to the design of the chemistry. Furthermore, the provided reagents do not require multiple purification steps as observed in other chemistries that increase laboratory complexity. In embodiments, the majority of the assay runtime comprises hands-off processes that include 4-24-hour hybridization, i.e., the binding of the gDNA to the synthetic oligonucleotides that targets the genomic regions of interest, and a 24-hour run processing time on the sequencing instrument.

All of the systems include multiple quality control metrics to ensure that the system is transferable. Advantageously, each laboratory’s test assay performs differently across different end users and there will be some variability of the output data. To ensure that the platform can account for these variables, the assay performance has been validated with strong baseline metrics based on industry standards to ensure “user” error vs “assay performance” error is identified. The overall wet-lab design of the test assay, stratifying into a simple workflow with less hands-on technologist time, minimizes error rate.

In embodiments, the method utilizes a set of oligonucleotides for screening for carrier or hereditary cancer gene variants, comprising at least one pair of oligonucleotides selected from Table 1 or Table 2. In embodiments, the pair of oligonucleotides comprises a forward primer and a reverse primer. In another embodiment, the method utilizes a set of oligonucleotides configured to amplify in an amplification reaction a nucleic acid sequence in a sample to generate an amplification product that can be sequenced utilizing next generation sequencing. In embodiments, the oligonucleotide is labelled. In embodiments, the oligonucleotide is fluorescently labelled. In embodiments, the oligonucleotide comprises a sequence tag. In embodiments, the method utilizes a tagged oligonucleotide probe comprising a sequence having at least 70%, 80%, 85%, 90%, 95%, 96%, 97%, 98%, or 99% sequence identity to a sequence selected from SEQ ID NOs: 1-87670 and a label. In embodiments, the method utilizes a tagged oligonucleotide probe comprising a sequence selected from SEQ ID NOs: 1-87670 and a label.

In embodiments, the set of oligonucleotides comprises about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the oligonucleotide pairs in Table 1 or Table 2. In embodiments, the set of oligonucleotides comprises all of the oligonucleotide pairs in Table 1 or Table 2.

In embodiments, the set of oligonucleotides comprises about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the oligonucleotide pairs in Table 1. In embodiments, the set of oligonucleotides comprises all of the oligonucleotide pairs in Table 1. In embodiments, the set of oligonucleotides comprises SEQ ID NOs: 1-59438. In embodiments, the oligonucleotides pairs provided herein can be used to decipher variants, known and de novo. In embodiments, the oligonucleotide pairs provided herein can detect wild-type and mutated variants.

In embodiments, the set of oligonucleotides comprises about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the oligonucleotide pairs in Table 2. In embodiments, the set of oligonucleotides comprises all of the oligonucleotide pairs in Table 2. In embodiments, the set of oligonucleotides comprises SEQ ID NOs: 59439-87670.

In embodiments, the panel includes autosomal and X-linked recessive carrier genes. In embodiments, the panel includes about 50 to about 200 autosomal and X-linked recessive carrier genes. In embodiments, the panel includes about 75 to about 100 autosomal and X-linked recessive carrier genes. In embodiments, the panel includes about 75 to about 150 autosomal and X-linked recessive carrier genes. In embodiments, the panel includes about 75 to about 200 autosomal and X-linked recessive carrier genes. In embodiments, the panel includes about 100 to about 200 autosomal and X-linked recessive genes. In embodiments, the panel includes 155 autosomal and X-linked recessive carrier genes.

In embodiments, the panel includes autosomal dominant oncology genes. In embodiments, the panel includes about 50 to about 200 autosomal dominant oncology genes. In embodiments, the panel includes about 75 to about 100 autosomal dominant oncology genes. In embodiments, the panel includes about 75 to about 150 autosomal dominant oncology genes. In embodiments, the panel includes about 75 to about 200 autosomal dominant oncology genes. In embodiments, the panel includes about 100 to about 200 autosomal dominant oncology genes. In embodiments, the panel includes 85 autosomal dominant oncology genes. In embodiments, the panel includes 85 autosomal dominant oncology genes.

In embodiments, the panel includes about 25, about 50, about 75, about 80, about 90, about 100, about 125, about 150, about 175, about 200, or about 300 autosomal and X-linked recessive carrier genes. In embodiments, the panel includes about 25, about 50, about 75, about 80, about 90, about 100, about 125, about 150, about 175, about 200, or about 300 autosomal dominant oncology genes.

In embodiments, the set of oligonucleotides comprises oligonucleotide probes directed toward at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or all of the carrier genes identified in Table 3. In embodiments, the set of oligonucleotides comprises oligonucleotide probes directed toward at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or all of the hereditary cancer genes identified in Table 3.

### Exemplary Cancer Screening Techniques And/or Genetic Testing Machine Learning Techniques

Provided here are non-limiting examples of methods for detecting carrier or cancer gene variants in a subject (which may, in some embodiments, be performed by the secure gateway computing device 103), the method comprising: performing a nucleic acid amplification assay on a sample from the subject using a set of oligonucleotides, comprising at least one oligonucleotide probe pair selected from Table 1 or Table 2, wherein the oligonucleotide probe pair is configured to amplify in an amplification reaction a gene region of interest; sequencing the amplified gene region of interest using next generation sequencing; and optionally generating an output corresponding to the sequenced gene region of interest. In embodiments, the output is raw sequencing data. In embodiments, the raw sequencing data is provided to a bioinformatics pipeline for analysis. The probe pairs in each of Tables 1 and 2 consist of a “forward” and “reverse” sequence listed, in each of the right column and the left column, respectively. For example, pair 1 of Table 1 consists of SEQ ID NO: 1 and SEQ ID NO: 29720. Pair 14861 of Table 1 consists of SEQ ID NO: 14861 and SEQ ID NO: 44580.

Padlock probes have been used to genotype a number of single nucleotide polymorphisms (SNPs). In embodiments, provided herein is a method of using padlock probes for full exon sequencing. In embodiments, provided herein are padlock probes that comprise a sequence selected from SEQ ID NOs: 1-59438. In embodiments, provided herein are padlock probes that comprise a sequence selected from SEQ ID NOs: 59439-87670. In embodiments, provided herein are padlock probes that comprise a probe pair selected from Table 1 or Table 2.

In embodiments, described herein is a method of performing a nucleic acid amplification assay comprising: extracting gDNA from a sample from a subject using an extraction kit; combining the gDNA with a plurality of oligonucleotide probe pairs in Tables 1 and 2; allowing time for hybridization; and loading the sample onto a NGS sequencer. In embodiments, the method further comprises: providing the raw sequencing data produced by the NGS sequencer to a bioinformatics pipeline for analysis. In embodiments, the hybridization time is about 24 hours. In embodiments, the NGS sequencer runtime is about 24 hours. In embodiments, the oligonucleotide probe pairs can be used with PCR-based amplification assays.

### Kit

In embodiments, described herein is a kit for screening for carrier or hereditary cancer gene variants, the kit comprising a plurality of oligonucleotide probe, along with the apparatus for analyzing data obtained from amplification of target sequences. In embodiments, the kit further comprises instructions for performing a method provided herein.

In embodiments, the kit may also include additional reagents necessary for performing the amplification and/or sequencing reactions, including polymerase enzymes, dNTPs, ddNTPs, and appropriate buffers. These additional reagents may be packaged separately or in combination.

### ASPIRA Synergy Genetics (ASG)

ASG is the first AI-based solution in characterizing variant-disease association that is fully automated for hereditary diseases. The solution can reduce analysis time significantly allowing customers to implement and run genetic tests at scale at a reduced cost, time and labor.

ASG is the first all-in-one genetic testing technology transfer solution from sample collection to customized genetic reporting of hereditary diseases. This would provide laboratories with the ability to internalize this testing modality, for the first time, as a fully encompassed technology transfer. Using ASG, the service labs would be able to offer new tests (products) to their customers with minimal investment and minimal risk, as a “plug-and-play” solution.

The offering includes front-end wet lab components and processes developed by ASPIRA which are customized to the cloud-based, end-to-end pipeline. Once specifically customized for the ASPIRA front-end, the pipeline will be powered by novel AI, and the entire analysis and interpretation process is fully automated, thereby reducing analysis time and labor.

Due to the sophistication of state-of-the-art technology and overhead costs, many clinical practices cannot implement genetic testing internally. As such, high complexity genetic tests, including carrier screen and hereditary cancer testing, have been outsourced by hospitals and small/medium regional laboratories to large commercial labs. There are several problems with this. The first is the cost; outsourcing the tests incurs higher costs. The second is that the hospitals and small/medium regional laboratories loses precious data which it can use and leverage internally, as the large commercial labs do not share the raw data, only the final report. In addition, the hospitals and small/medium regional laboratories are limited to the scope of the provided tests, and lack the ability to influence it. Moreover, the hospitals and small/medium regional laboratories cannot build their own expertise in these tests and leverage their existing human capital.

In order for a lab to offer a test similar to the larger established genetic testing laboratories, it would require a significant investment of hiring a large staff of full-time employees with specific training and expertise in several specialties, including; bioinformatics, data science, CLIA laboratory testing, and molecular genetics, as well as being clinically boarded by ACMG to support such a platform. The main requirements are:

Sequencing platform - selecting the most appropriate targeting chemistry platform along with the exact design and workflow which should be used to detect all required variants. This requires well-trained assay development PhDs who can design the in-silico genomic targets to ensure proper coverage in the region of interest.

Data Analysis platform - while some sequencing platforms today provide means for analyzing the raw data, they are not enough to meet the analysis requirements for achieving the test analytical requirements and so an analytics platform is needed with personnel that are trained to analyze large datasets from sequencing files.

Clinical Data Curation - curating the exact scope of genes and diseases which should be performed as part of the test, as well as curating known/prevalent variants. In addition, for each disease, clinical information should be curated, including disease information, prevalence, detection rate etc. which are needed for generating the final report for the patient. This would require MD/PhD trained, and clinical board-certified molecular geneticists by the American Board of Medical Genetics (ABMG), as well as certified genetic counselors to review and manage the curation process.

Reporting and workflow system - in order to support test scale, a software solution would be needed to manage the test workflow and to provide the final report which should be provided to the end-user (referring physician / patient). An automated reporting platformwould need to be designed with a software engineer or raw data would need to be instituted into an already existing medical record platform.

Validations - once all the above is in place, validation should be carried out. This would include purchasing positive control samples, design validation experiments and actually sequencing and validating the assay and workflow. In order to properly validate a molecular genetic test, personnel with training in molecular genetics and CLIA/LDT validation experience are required.

Creating such a complex system and workflow supported by a large staff, requires significant time, money, and additional resources, making the barrier entry too high for most of the labs today. Due to that, only large commercial labs that can manage the complexities of establishing a genetics lab and maintaining dynamic interpretation of the results offer these kinds of tests.

The complexities around maintaining an enriched database of known variants and keeping up with daily published findings on changes to gene/variant interpretation can be unattainable to manage at a small scale. In combination with complex technology and progressing to consistently optimize the genetic test offering, institutions cannot internalize a genetic testing offering

The subject matter described herein addresses these issues and more. The compositions, kits and methods for genetic testing disclosed herein can provide laboratories with a simple wet lab solution with a limited number of steps that shortens the chemistry and reduces turnaround time which in turn helps laboratories internalize genetic testing. Since genetic testing is dynamic in nature and new variants and genes are constantly being re-characterized and associated with disease, the subject matter provided herein can allow offering a competitive and clinically relevant genetic test to patients without having to manage these changes. This allows the assays to stay clinically relevant without having to build out the infrastructure, while leveraging AI to provide “live” reinterpretation of genetic diseases and consistently develop panels that support clinical management and fit the clinicians needs. The following features make the ASG product unique.

Simplified and Unified Workflow - A simple wet lab solution with a limited number of steps, shortens the chemistry by a day which allows a reduced turnaround time (TAT). Most importantly the wet lab bench work is “Easy to teach and implement”.

AI-based interpretation capabilities and full automation - ASG AI-based Interpretation Engine automates the process and allows handling the scale of the test potential findings, thereby reducing the time and labor required for creating the final clinical report.

Single platform - ASG has a single unified workflow for all genes and diseases compared to multiple different technologies / workflows at most companies, due to the complexity of the human genome. This is accomplished by unique algorithmic capabilities powered by analytical bioinformatics. In addition, both carrier and hereditary cancer in the same technology transfer are provided.

End-to-end Process - gDNA to a customized clinical report. ASG provides a customized clinical and scientific interpretation of the patients sequencing data into a clinically actionable report, saving time and cost.

### Data Analysis Backend: Variant Calling Pipeline

The human genome is complex and there is no single method for detecting all variants. Due to these challenges, genetic testing workflows at most labs require several different technologies and workflows. However, ASG has a single workflow which is built using unique algorithms powered by an analytical bioinformatics pipeline. This includes detection of short variants (SNPs/Indels), Copy Number Variants (CNVs), and accurately detecting variants in challenging regions such as genes with known pseudogenes or homologous regions - SMA, GBA, HBA1/HBA2, CYP21A2.

As part of the development of ASG, the analytical pipeline applies a variety of workflows for detecting all variant types and meet the analytical challenges associated with them.

For genes with pseudogenes and homologous regions, a dedicated solution is created, built upon established graph-based aligner software and AI callers.

Structural Variant Classification and Verification:

CNVs: The ASG bioinformatics platform employs a dedicated algorithm to systematically identify CNVs in whole-exomes with robust analytical performance. The algorithm was developed specifically for whole exome sequencing for hybridization-based capture. It employs a machine-learning based, anomaly detection algorithm, in which variants are determined based on an exon-level coverage.

As part of ASG, an algorithm would be tailored specifically to the test in order to enable high confidence detection of deletion and duplication more than one exon heterozygote resolution and up to whole gene or large clusters, in order to achieve clinical-grade analytical performance. This is done by algorithms dedicated to the chemistry used in ASG. As part of it, the variant caller has been trained on positive and negative samples which have been confirmed using orthogonal methods. Special attention is given to genes in the panel in which CNVs are a common mechanism for the diseases e.g. in Duchenne (DMD) and Cystic Fibrosis (CFTR). The end goal would be that for genes covered in the test, sensitivity and specificity would be targeted to 100%. This is as opposed to the standard today in which CNV results have low specificity and thus many false positives results.

Pseudogenes (i.e. SMA): Spinal muscular atrophy (SMA) is an autosomal recessive disease in which the most common pathogenic variant is a deletion of exon 7 in the SMN1 gene. SMN1 and SMN2 genes are highly homologous and differ only by five nucleotides. The SMN1 gene is the only functional gene and mutations within this gene cause SMA, whereas the SMN2 is the ‘false gene’ or better known as the pseudogene due to the >95% nucleotide sequence homology. It is important to ensure that the test is able to decipher the ‘true’ gene, SMN1, from the pseudogene, SMN2, and subsequently is being deciphered by the bioinformatic pipeline to properly call a disease-causing deletion. Common methods for performing carrier detection are mostly being done using dedicated assays such as Multiple Ligation Probe Amplification (MLPA) or quantitative PCR (qPCR) that is readily able to decipher a large genomic loss known as a copy number (CN) change. Primarily, these methods are used alone or in addition to sequencing-based carrier tests, making it both tedious and more expensive due to the use of multiple assays.

In order to accurately detect carriers of SMA, a novel machine learning (ML) based algorithm is used for detection of carriers. The algorithm is based on several key technological developments, including ASG’s aligner and its AI-based variant caller. The combination of these technologies enables using the short-read NGS sequencing data for detecting variants in regions which tradtionally require additional assays.

As part of it, the ML model has been trained on positive and negative samples that were obtained from biobanks and existing in-house samples with known CN changes. The methods were thoroughly validated using orthogonal methods. Detecting SMA carriers by a single testing paradigm significantly reduces the test cost and complexities of running multiple orthogonal workflows in the laboratory.

### Data Analysis Backend: Interpretation Engine

Today, in the era of whole genome sequencing, genetic tests encompass almost all of the potential variants in a gene, thereby increasing the detection rate of disease and providing a more comprehensive report. This is distinct from previous methods, which primarily focused on a specific concise list of known variants. With that, the process of variant interpretation has become a time consuming and error prone process creating a significant bottleneck whereby interpreting case variants can take hours. In addition, it requires a massive in-house knowledgebase of evidence which requires manual curation by an experienced team.

ASG uses an AI-based interpretation engine. The engine dynamically assimilates evidence from hundreds of sources and databases to create a consolidated evidence-graph which allows automating the interpretation process and scaling it.

The AI-based interpretation engine is optimized and trained to support the ability to test - Expanded Carrier and Hereditary Cancer associated genes. The pipeline’s AI has been trained on known variants to accurately classify novel variants and to reduce the need for ad-hoc interpretation that can create reporting bottlenecks. While the final clinical report would be signed and reviewed by the service lab director, the AI-based genomic interpretation reduces the interpretation time to a minimum and removes analytical workflow from the customer as it is automatically built into the ASG pipeline.

Aspects of the disclosed subject matter are further described in the following nonlimiting Examples. It should be understood that these examples are given by way of illustration only.

### Example 1: Designing and Synthesis of the Oligonucleotide Probes for Carrier or Hereditary Cancer Screening

The most relevant genes/diseases to be included on the panels which are clinically actionable and have a carrier frequency higher than 1 in 500 across multiple ethnicities (i.e. Pan-ethnic panel) were identified. The testing panels have been designed considering well-established clinical guidelines from ACMG, newborn screening guidelines from American College of Obstetrics and Gynecology, ACOG expanded carrier testing and the genomic content assessed routinely in persons of Ashkenazi Jewish descent because of the increased carrier frequency in this population. Additionally, the genes of interest were mapped against ClinVar to ensure all clinically relevant variants with a minimum of 2 stars were included in the panel to increase disease detection rate. Using this exercise, we constructed clinically actionable and-disease relevant germline testing panels. One panel includes autosomal and X-linked recessive carrier genes. Another panel includes autosomal dominant oncology genes. An exemplary list of the genes and diseases covered are shown in Table 3 below. In embodiments, de novo gene variants can be deciphered using the panel.

In-silico genomic targets were identified to ensure proper coverage in the region of interest and to detect all required variants. A set of oligonucleotide probes were designed targeting a genomic region(s) and assigned efficiency scores consisting of, but not limited to: (1) presence of a guanine or cytosine as the 5′-most base of the ligation arm, (2) The number of dbSNP entries intersecting targeting arm sites, and (3) root squared deviation of the arms predicted melting temperatures from optimal values derived from empirical studies of capture efficiency. Using these efficiency metrics allowed for probe performance ranking and allowed ‘tiling’ across the region of interest (ROI) so that every genomic position is properly captured by multiple probes. Each probe is specifically designed for the selected list of targeted genes, i.e., Table 3, in order to properly sequence the required genes and variants of interest. The oligonucleotide probes were subsequently optimized for efficiency based on the ranking metrics listed above. Oligonucleotide probes were synthesized by standard methods.

The oligonucleotide pairs provided herein can be used to amplify the entire gene sequence including exonic, promoter, and splice-site regions. A reference sequence from the NIST GIAB (Genome in the Bottle) NA12878 is used to ensure target capture efficiency. The regions comprise an exon, a splice-site, and/or a promoter. Each oligonucleotide pair comprises a forward and reverse primer. The oligonucleotide pairs for SEQ ID NOs: 1-59438 (Carrier probes) and SEQ ID NOs: 54439-87670 (Cancer probes) can be found in Tables 1 and 2, respectively.

TABLE 3

| | | |
|---|---|---|
| Gene Target | Panel Type | Genetic Disorder |
| ABCCA8 | Carrier | Familial Hyperinsulinism ABCCA8- related (Diabetes mellitus, 3 types in neonate) |
| ABCD1 | Carrier | Adrenoleukodystrophy, X-L |
| ACAD9 | Carrier | Acyl-CoA dehydrongenase-9 (ACAD9) Deficiency |
| ACADM | Carrier | Medium-chain acyl-CoA dehydrogenase (MCAD) deficiency |
| ACADVL | Carrier | VCAD |
| ADA | Carrier | Adenosine deaminase def/SCIDS |
| ADAMTS2 | Carrier | Ehlers-Danlos Syndrome, Dermatospoaraxis TVIIC |
| AGL | Carrier | GSDTIII |
| AGXT | Carrier | Primary HyperoxaluriaT1 |
| ALPL | Carrier | Hypophosphatasia |
| AMT | Carrier | Glycine Encephalopathy |
| ARG1 | Carrier | Arginase Def |
| ARSA | Carrier | metachromatic leukodystrophy |
| ASL | Carrier | Argininosuccinate Lyase Deficiency |
| ASPA | Carrier | Canavan Disease |
| ASS1 | Carrier | Citrullinemia |
| ATP7B | Carrier | Wilson Disease |
| BBS2 | Carrier | Bardet-Biedel Syndrome |
| BCKDHA | Carrier | MSUD1A |
| BCKDHB | Carrier | MSUD1B |
| BTD | Carrier | Biotinidase Def |
| CBS | Carrier | Homocystinuria CBST def |
| CDH23 | Carrier | Usher Type 1D (USH1D) |
| CEP290 | Carrier | BBS, Joubert (Japanese, LCA, MS, SLS) |
| CERKL | Carrier | RP 26 |
| CFTR | Carrier | cystic fibrosis |
| CLN3 | Carrier | Neuronal ceriod lipofuscinosis, CLN3-related |
| CLN5 | Carrier | Neuronal ceriod lipofuscinosis, CLN5-related |
| CLN8 | Carrier | Neuronal ceriod lipofuscinosis, CLN8-related |
| CLRN1 | Carrier | Usher Syndrome Type 3A |
| CNGB3 | Carrier | Achromatopsia |
| COL27A1 | Carrier | Dystrophic epidermolysis bullosa |
| COL4A3 | Carrier | Alport Syndrome; Metachromatic leukodystrophy |
| COL4A4 | Carrier | Alport Syndrome; Metachromatic leukodystrophy |
| COL7A1 | Carrier | Dystrophic epidermolysis bullosa |
| CPT1A | Carrier | CPT IA |
| CPT2 | Carrier | Carnitine Palmitoyltransferase II Deficiency |
| CRB1 | Carrier | LCA |
| CTNS | Carrier | Cystinosis |
| CYP11B1 | Carrier | Congenital adrenal hyperplasia due to 12-hydroxlase deficiency |
| CYP1B1 | Carrier | CAH |
| CYP21A2 | Carrier | Congenital adrenal hyperplasia due to 12-hydroxlase deficiency |
| CYP27A1 | Carrier | Cerebrotendinous Xanthomatosis |
| DHCR7 | Carrier | SLOS |
| DHDDS | Carrier | Retinitis pigmentosa, nonsyndromic |
| DLD | Carrier | Maple Syrup Disease, Dihydrolipamide dehydrogenase deficiency |
| DMD | Carrier | Duchenne Muscular Dystrophy |
| DNAI2 | Carrier | Primary ciliary dyskinesia |
| ELP1 (IKBKAP) | Carrier | Familial dysautonomia |
| ETFDH | Carrier | Glutaric Aciduria II |
| ETHE1 | Carrier | Ethylmalonic encelophathy/ Leigh Syndrome (80% ntDNA) |
| EYS | Carrier | Retinitis Pigmentosa 25 |
| F11 | Carrier | Factor XI deficiency |
| FAH | Carrier | Tyrosinemia T1 |
| FANCA | Carrier | Fanconi Anemia |
| FANCG | Carrier | Fanconi Anemia |
| FH | Carrier | Fumarase deficiency |
| FKRP | Carrier | FKRP-related disorders (including Walker-Warburg) |
| FKTN | Carrier | FKTN-related Disorders |
| G6PC | Carrier | Glycogen storage disease Ia |
| GAA | Carrier | Pompe Disease |
| GALC | Carrier | Krabbe Disease |
| GALK1 | Carrier | Galactokinase deficiency with cataracts |
| GALNS | Carrier | Mucopolysaccharidosis IVA |
| GALT | Carrier | Galactosemia |
| GAMT | Carrier | Cerebral creatine deficiency syndrome 2 |
| GBA | Carrier | Gaucher disease, type I |
| GBE1 | Carrier | Glycogen Storage Disease IV |
| GCDH | Carrier | Glutaricaciduria, type I |
| GJB1 | Carrier | Charcot-Marie-Tooth neuropathy, X-linked dominant, 1 |
| GJB2 | Carrier | Non-syndromic hearing loss, GJB2-disease |
| GLB1 | Carrier | GM1-gangliosidosis, type I |
| GLDC | Carrier | Glycine encephalopathy |
| GNPTAB | Carrier | Mucolipidosis II alpha/beta |
| GNPTG | Carrier | Mucolipidosis III gamma |
| GNS | Carrier | Mucopolysaccharidosis type IIID |
| GUSB | Carrier | Mucopolysaccharidosis VII |
| HADHA | Carrier | Fatty liver, acute, of pregnancy, HELLP syndrome, maternal, of pregnancy, LCHAD deficiency |
| HAX1 | Carrier | Neutropenia, severe congenital 3, autosomal recessive |
| HBA1 | Carrier | alpha Thalassemia |
| HBA2 | Carrier | Thalassemia, alpha- |
| HBB | Carrier | beta Thalassemia |
| HEXA | Carrier | Tay-Sachs disease |
| HLCS | Carrier | Holocarboxylase synthetase deficiency |
| HMGCL | Carrier | HMG-CoA lyase deficiency |
| HOGA1 | Carrier | Primary hyperoxaluria type 3 |
| HPS1 | Carrier | Hermansky-Pudlak syndrome 1 |
| HPS3 | Carrier | Hermansky-Pudlak syndrome |
| HSD17B4 | Carrier | D-bifunctional protein deficiency |
| IDUA | Carrier | Muccopolysaccharidos, type I (Hurler) |
| IVD | Carrier | Isovaleric acidemia |
| KCNJ11 | Carrier | Congenital hyperinsulinism; Permanent neonatal diabetes mellitus |
| LAMA2 | Carrier | Muscular dystrophy, congenital, merosin deficient or partially deficient |
| LAMB3 | Carrier | Familial hypercholesterolemia |
| LHX3 | Carrier | Pituitary hormone deficiency, combined, 3 |
| LIFR | Carrier | Stuve-Wiedemann syndrome/Schwartz-Jampel type 2 syndrome |
| LIPA | Carrier | Lysosomal acid lipase deficiency |
| LOXHD1 | Carrier | Nonsyndromic hearing loss |
| LRPPRC | Carrier | Leigh syndrome with Complex IV deficiency |
| LYST | Carrier | Chediak-Higashi syndrome |
| MAN2B1 | Carrier | Alpha-mannosidosis |
| MCCC1 | Carrier | 3-Methylcrotonyl-CoA carboxylase 1 deficiency (3-MCC deficiency) |
| MCCC2 | Carrier | 3-Methylcrotonyl-CoA carboxylase 2 deficiency (3-MCC deficiency) |
| MCOLN1 | Carrier | Mucolipidosis type IV i |
| MED17 | Carrier | Postnatal Progressive Microencephaly with Seizures and Brain Atrophy |
| MEFV | Carrier | Familial Mediterranean fever* |
| MESP2 | Carrier | Spondylocostal dysostosis |
| MFSD8 | Carrier | Neuronal Ceroid-Lipofuscinosis, MFSD8-Related |
| MKS1 | Carrier | Joubert syndrome 28; Meckel syndrome 1; Bar- det-Biedl syndrome 13 |
| MLC1 | Carrier | Megalencephalic leukoencephalopathy with subcortical cysts |
| MMAA | Carrier | Methylmalonic aciduria, cblA type |
| MMAB | Carrier | Methylmalonic aciduria, cblB type |
| MMACHC | Carrier | Mehylmalonic acidemia and homocystinuria, cbID type |
| MPL | Carrier | Congenital amegakaryocytic thrombocytopenia |
| MPV17 | Carrier | Hepatocerebral mitochondrial DNA depletion syndrome, MPV17-related |
| MTTP | Carrier | Abetalipoproteinemia |
| MUT | Carrier | Methylmalonic acidemia, MUT-related |
| MYO7A | Carrier | Usher T1B (USH1B) |
| NAGLU | Carrier | Mucopolysaccharidosis type IIIB (Sanfilippo syndrome B) |
| NDRG1 | Carrier | Charcot-Marie-Tooth disease, type 4D |
| NDUFAF5 | Carrier | Mitochondrial complex I deficiency (Leigh Syndrome) |
| NDUFS6 | Carrier | Mitochondrial complex I deficiency (Leigh Syndrome) |
| NEB | Carrier | Nemaline myopathy |
| NPC1 | Carrier | Niemann-Pick disease, type C1 |
| NPC2 | Carrier | Niemann-pick disease, type C2 |
| NPHS1 | Carrier | Congenital nephrotic syndrome, type 1 |
| NPHS2 | Carrier | Congenital nephrotic syndrome, type 2 |
| NR2E3 | Carrier | Enhanced S-cone syndrome; Retinitis pigmentosa 37 |
| NTRK1 | Carrier | Congenital insensitivity to pain with anhidrosis |
| PAH | Carrier | Phenylalanine hydroxylase deficiency (Pheylketonuria) |
| PCCA | Carrier | Propionicacidemia |
| PCCB | Carrier | Propionicacidemia |
| PCDH15 | Carrier | Non-syndromic hearing loss, PCDH15-related: Usher Syndrome, type 1F |
| PEX1 | Carrier | Zwelleger Disease |
| PEX2 | Carrier | Zwelleger Disease |
| PEX6 | Carrier | Zwelleger Disease |
| PEX7 | Carrier | Rhizomelic chondrodysplasia punctata, type 1 |
| PHGDH | Carrier | 3-phosphoglycerate dehydrogenase deficiency |
| PKHD1 | Carrier | Polycystic kidney disease 4, with or without hepatic disease |
| PMM2 | Carrier | Congenital disorder of glycosylation, type Ia |
| RPGRIP1L | Carrier | COACH syndrome; Joubert syndrome 7; Meckel syndrome 5 |
| RTEL1 | Carrier | Dyskeratosis congenital type 5 |
| SLC22A5 | Carrier | Carnitine deficiency, systemic primary |
| SLC25A15 | Carrier | Hyperornithinemia-hyperammonemia-homocitrullinemia syndrome |
| SLC35A3 | Carrier | Arthrogryposis, MR and seizures |
| SMN1 | Carrier | Spinal muscular atrophy |
| SMPD1 | Carrier | Niemann Pick Type A/B |
| TMEM216 | Carrier | Joubert Syndrome |
| USH1C | Carrier | Usher type 1C related disorders (USH1C) |
| USH2A | Carrier | Ushers Syndrome |
| ATM | Carrier/Hereditary Cancer | Ataxia Telangiectasia, BC 17-52% risk, OC increased risk |
| BLM | Carrier/Hereditary Cancer | Bloom |
| FANCC | Carrier/Hereditary Cancer | Fanconi anemia, complementation group C; BC |
| NBN | Carrier/Hereditary Cancer | Nijmegen breakage syndrome |
| RAD51C | Carrier/Hereditary Cancer | Fanconi anemia, BC, Ovarian up to 10% |
| WT1 | Hereditary Cancer | Wilms Tumor Syndrome |
| AIP | Hereditary Cancer | of familial isolated pituitary adenoma (FIPA) |
| ALK | Hereditary Cancer | familial neuroblastoma, and confer a small increased risk (low penetrance) for this type of cancer. |
| APC | Hereditary Cancer | CRC |
| AXIN2 | Hereditary Cancer | CRC |
| BAP1 | Hereditary Cancer | Renal/Urinary/ BAP1 tumor predisposition syndrome (BAP1-TPDS). |
| BARD1 | Hereditary Cancer | HBOC |
| BMPR1A | Hereditary Cancer | Juvenile Polyposis syndrome, hereditary; CRC, Gastric |
| BRCA1 | Hereditary Cancer | HBOC |
| BRCA2 | Hereditary Cancer | HBOC |
| BRIP1 | Hereditary Cancer | HBOC |
| CASR | Hereditary Cancer | Pancreatic Cancer; AD hypocalcemia, familial hypocalciuric hypercalcemia, neonatal severe hyperparathyroidism |
| CDC73 | Hereditary Cancer | Hyperparathyroidism-jaw tumor syndrome, which increases the risk for renal tumors (hamartomas, Wilms tumor), parathyroid tumors, and ossifying fibromas of the maxilla or mandible. |
| CDH1 | Hereditary Cancer | HBOC |
| CDK4 | Hereditary Cancer | melanoma and possibly for pancreatic cancer |
| CDKN1B | Hereditary Cancer | MEN4 |
| CDKN1C | Hereditary Cancer | maternally inherited, pathogenic variants in the CDKN1C gene are one cause of Beckwith-Wiedemann syndrome, which is associated with an increased risk for embryonal tumors, including Wilms tumor, hepatoblastoma, neuroblastoma, and rhabdomyosarcoma, as well as other clinical conditions. |
| CDKN2A | Hereditary Cancer | HBOC |
| CEBPA | Hereditary Cancer | Myeloid malignancies |
| CHEK2 | Hereditary Cancer | Li-Frauemeni, HBOC |
| CTNNA1 | Hereditary Cancer | MEN4 |
| EGFR | Hereditary Cancer | Lung Cancer |
| EPCAM | Hereditary Cancer | Uterine, CRC (70%), Gastric, Pancreatic, Prostate |
| FH | Hereditary Cancer | Hereditary Leiomyomatosis and Renal Cell Cancer (HLRCC). The evidence for an association between HLRCC and paragangliomas or pheochromocytomas is contradictory, additional research is needed |
| FLCN | Hereditary Cancer | Birt-Hogg-Dube syndrome which is characterized by cutaneous manifestations, spontaneous pneumothorax and renal tumors, including renal cell carcinoma |
| GATA2 | Hereditary Cancer | GATA2 deficiency and Emberger syndromes, both of which increase an individual’s risk for myelodysplasia and acute myeloid leukemia. |
| GPC3 | Hereditary Cancer | X-linked loss of function pathogenic variants in the GPC3 gene have been associated with type 1 Simpson-Golabi-Behmel syndrome which is an overgrowth syndrome that may include multiple congenital anomalies including intellectual disability, distinctive craniofacial features, organomegaly, and an increased risk of embryonal tumors, including Wilms tumor, hepaoblastoma and hepatocellular carcinoma, among others. |
| GREM1 | Hereditary Cancer | CRC |
| HOXB13 | Hereditary Cancer | Prostate Cancer 60% |
| HRAS | Hereditary Cancer | Costello syndrome. Individuals with Costello syndrome have a 15% lifetime risk for developing a malignant tumor, with rhabdomyosarcomas occurring most frequently. |
| KIT | Hereditary Cancer | Gastric Cancer |
| MAX | Hereditary Cancer | Associated with susceptibility to pheochromocytoma and paraganglioma. Risk for the development of a pheochromocytoma in individuals with germline MAX pathogenic variants is much higher if the variant was paternally inherited. |
| MEN1 | Hereditary Cancer | Multiple Endocrine Neoplasia, Pancreatic Cancer |
| MET | Hereditary Cancer | Rare cases of familial papillary renal cell carcinoma, although additional studies are needed given the small number of reported families. |
| MITF | Hereditary Cancer | Associated with an increased risk for renal cell carcinoma. However, additional studies are needed. |
| MLH1 | Hereditary Cancer | Lynch syndrome |
| MRE11A | Hereditary Cancer | Predisposition to breast cancer. Biallelic mutations in the MRE11A gene are associated with MRE11 deficiency, an ataxia telangiectasia-like disorder. |
| MSH2 | Hereditary Cancer | Lynch syndrome |
| MSH3 | Hereditary Cancer | autosomal recessive MSH3-associated polyposis; CRC |
| MSH6 | Hereditary Cancer | Lynch syndrome |
| MUTYH | Hereditary Cancer | MYH-assoc polyposis |
| NF1 | Hereditary Cancer | Neurofibromatosis, BC |
| NF2 | Hereditary Cancer | Neurofibromatosis, BC |
| NTHL1 | Hereditary Cancer | Familial adenomatous polyposis-3 (FAP3) which is also referred to as NTHL1-associated polyposis (NAP). |
| PALB2 | Hereditary Cancer | HBOC |
| PDGFRA | Hereditary Cancer | Polyps, multiple and recurrent inflammatory fibroid, gastrointestinal |
| PHOX2B | Hereditary Cancer | familial neuroblastoma |
| PMS2 | Hereditary Cancer | HNPCC |
| POLD1 | Hereditary Cancer | Colorectal cancer and other adenomas including endometrial and breast. |
| POLE | Hereditary Cancer | Early onset colorectal cancer (CRC) and polyposis, also known as Polymerase Proofreading-associated Syndrome (PPAS). Further studies are needed to determine which cancers are directly related to POLE gene variants and the levels of associated risks. Based on the information available today, the risk for colorectal cancer appears to be significantly elevated, and the risk for brain tumors may also be increased . |
| POT1 | Hereditary Cancer | Increased risk for melanoma and gliomas |
| PRKAR1A | Hereditary Cancer | Carney Complex. Individuals with this condition are at approximately a 10% risk to develop a schwannoma, in addition to other clinical findings. |
| PTCH1 | Hereditary Cancer | Nevoid Basal Cell Carcinoma syndrome (NBCSS), which increases the risk for medulloblastoma. For individuals with NBCSS caused by PTCH1 variants, this risk, though, is less than 2%. |
| PTEN | Hereditary Cancer | PTEN hamartoma tumor syndrome |
| RAD50 | Hereditary Cancer | HBOC - early evidence |
| RAD51D | Hereditary Cancer | BC, Ovarian up to 19% |
| RB1 | Hereditary Cancer | Increased risk for retinoblastoma, melanoma, and osteo-and soft tissue sarcomas. Individuals with biallelic variants are more severely affected than those who are heterozygous. |
| RECQL4 | Hereditary Cancer | Rothmund-Thomson syndrome (RTS), Baller-Gerold syndrome (BGS), and RAPADILINO syndrome. Individuals with these conditions are at an increased risk for various cancers, including osteosarcoma, basal cell carcinoma, squamous cell carcinoma, and lymphoma. |
| RET | Hereditary Cancer | Multiple endocrine neoplasia type 2 (MEN 2), which is associated with medullary thyroid carcinoma, pheochromocytoma, and other clinical findings. |
| RUNX1 | Hereditary Cancer | Familial platelet disorder and an increased risk for myeloid malignancies. |
| SCG5 | Hereditary Cancer | CRC |
| SDHA | Hereditary Cancer | One genetic cause of Hereditary Paragangliomas-Pheochromocytoma Syndromes (HPPS), and are responsible for approximately .6-3% of cases |
| SDHAF2 | Hereditary Cancer | Susceptibility to paragangliomas. Risk for developing a paraganglioma in individuals with germline SDHAF2 pathogenic variants is much higher if the variant was paternally inherited, and they most frequently occur in the skull base and neck. |
| SDHB | Hereditary Cancer | Cowden/CRCHereditary Paraganglioma-Pheochromocytoma syndrome (HPPS) and are responsible for approximately 22-38% of cases. They are also associated with Carney-Stratakis syndrome, which is characterized by the presence of paragangliomas and gastrointestinal stromal tumors. SDHB-related HPPS has the highest risk for malignancy in comparison to the different genetic causes of the condition. |
| SDHC | Hereditary Cancer | 4 and 8% of cases of Hereditary Parganglioma-Pheochromocytoma syndrome (HPPS). |
| SDHD | Hereditary Cancer | Pheochromocytoma-paraganglioma syndrome, CRC |
| SMAD4 | Hereditary Cancer | CRC 38-65%, 21% risk Gastric |
| SMARCA4 | Hereditary Cancer | Rhabdoid tumor predisposition syndrome 2; elevated ovarian/endometrial |
| SMARCB1 | Hereditary Cancer | Rhabdoid tumor predisposition syndrome 1 (RTPS1), schwannomatosis, and Coffin-Siris syndrome |
| SMARCE1 | Hereditary Cancer | Familial meningioma, Coffin-Siris syndrome |
| STK11 | Hereditary Cancer | Peutz-Jeghers Syndrome, 45-50% BC, 18-21% OC, 9% Uterine, 39% CRC, 29% Gastric, 11-36% Pancreatic |
| SUFU | Hereditary Cancer | Nevoid basal cell Carcinoma syndrome (NBCCS; also called Gorlin syndrome). While a few cases of rhabdomyosarcoma have been reported in individuals with NBCCS, additional research is needed to confirm |
| | | this association given the small number of reported cases. |
| TERC | Hereditary Cancer | Dyskeratosis congenita, which is associated with an increased risk for bone marrow failure, myelodysplastic syndrome, and leukemia. |
| TERT | Hereditary Cancer | Dyskeratosis congenita, which is associated with bone marrow failure, myelodysplastic syndrome, and leukemia. Patients with autosomal dominant (heterozygous) TERT pathogenic variants tend to have milder disease than those with the autosomal recessive (biallelic) form. |
| TMEM127 | Hereditary Cancer | Pheochromocytomas. |
| TP53 | Hereditary Cancer | Li-Frauemeni, BC up to 79% risk |
| TSC1 | Hereditary Cancer | Renal/Urinary Cancer |
| TSC2 | Hereditary Cancer | Renal/Urinary Cancer |
| VHL | Hereditary Cancer | Von-Hippel Lindau Syndrome/ Cerebellar hemangioblastoma, Pancreatic cysts, Spinal hemangioblastoma, Retinal capillary hemangioma |
| WRN | Hereditary Cancer | Werner syndrome, which is characterized by features of premature aging that includes an increased risk for many types of cancer. At this time, heterozygous carriers of a pathogenic variant in WRN have not been shown to have an increased risk of cancer. |

### Example 2: Validation of the Assays

Validation and verification of the assays were performed on an Illumina sequencing instrumentation (NextSeq 550 and HiSeq2500).

The analytical pipeline was customized to accurately perform alignment and variant calling using targeted sequencing data generated from the dedicated capture kit. This included: customization of the alignment process, customization of the short variant calling, both SNPs and indels, customization and development of the copy number variant caller.

Homologous genes pipeline development: Expanded carrier testing includes several genes with homologous regions, either other genes or pseudogenes (SMA, GBA and others). In order to properly call variants in these genes, a dedicated pipeline was developed based on dedicated algorithms.

An analytical verification for carrier screening genes was performed for the carrier assay in order to make sure it met the quality requirements, and final adjustments in chemistry and the analytical pipeline were made. The assay was verified using positive control samples which were detected using orthogonal methods (e.g., data from Coriell biobank). This ensured that positive results were accurately called and that there were no false positive results. As part of this step 3 sequencing runs were performed in order to detect probe design issues, remove possible “batch” effects, and determine baseline metrics of assay performance.

A formal blinded analytical validation for carrier screening was performed. This was achieved by doing three separate experiments in a blinded protocol. First, a round of blinded validation runs of unique samples to meet the NGS test standards was performed to determine the analytical validity of the assay, negative and positive predictive values, performance and accuracy of the assay and a final validation was performed to meet the NGS test standards. Sample replicates were run within and across experiments to determine inter-run and intra-run reproducibility.

Similarly, analytical verification for a hereditary cancer gene assay was also performed. The assay was verified using positive control samples which were detected using orthogonal methods (e.g., data from Coriell biobank) was performed. This verified that positive results were accurately called and that there were no false positive results. Multiple sequencing runs, i.e., at least 3 were performed in order to detect and address issues in the design of the probes, and to be able to remove “batch” effects and determine baseline metrics of assay performance.

Likewise, a formal blinded analytical validation of the assay was performed for hereditary cancer genes. Three experiments in a blinded protocol were performed. First, a blinded validation run of unique samples to meet the NGS test standards and determine the analytical validity of the assay, negative and positive predictive values, performance and accuracy of the assay and a final validation was performed to meet the NGS test standards. Sample replicates were run within and across experiments to determine inter-run and intra-run reproducability.

### Example 3: Running the Assays

Genomic DNA (gDNA) is extracted from a sample from a subject using an extraction kit. The gDNA is combined with the oligonucleotide probe pairs in Table 1 or 2 and allowed to hybridize for 16-24 hours.

Amplification reagents are added and the final targeted library is pooled and loaded onto a NGS sequencing instrument.

The resulting sequence data from the NGS sequencer is provided to the bioinformatics platform for variant calling. The bioinformatics platform detects and reports SNVs, indels, CNVs, homologous regions, and pseudogenes of carrier and hereditary cancer genes including, for example, the genes listed in Table 3.

### Reporting Service

Once the service lab’s patient(s) data is analyzed, the ASG platform creates a final clinical report, along with the main findings. The ASG platform provides the full set of tools supporting the lab workflow for reviewing the results, confirming and storing them for backup and regulatory requirements.

Similar to the analysis process, the reporting process is fully automated in order to support large scale testing. In an embodiment, a genetic scientist reviews the report content prior to delivering to the service lab as a recommended, “pre-signed” clinical report. Once the report is received by the service lab, their lab director reviews the content as incorporated in the report and finalizes all content with their signature, to generate a “Final-signed” clinical report.

### HIPAA and Privacy Laws

In order to address HIPAA and privacy laws, the ASG platform has a number of features. In embodiments, to ensure that the ASG portal protects personal history information (PHI) strict components have been built into the domain. In embodiments, all communication between any of the components (frontend, API, backend services) is done using HTTPS using TLS 1.2 protocol to encrypt data. In embodiments, the entire ASG platform is segmented into several independent private networks with access-control list (ACL) and routing filtering. In embodiments, the data stored in the DB is encrypted at rest and in flight. In embodiments, all incoming traffic is going through various port security groups as well as a Web Application Firewall to actively filter incoming traffic

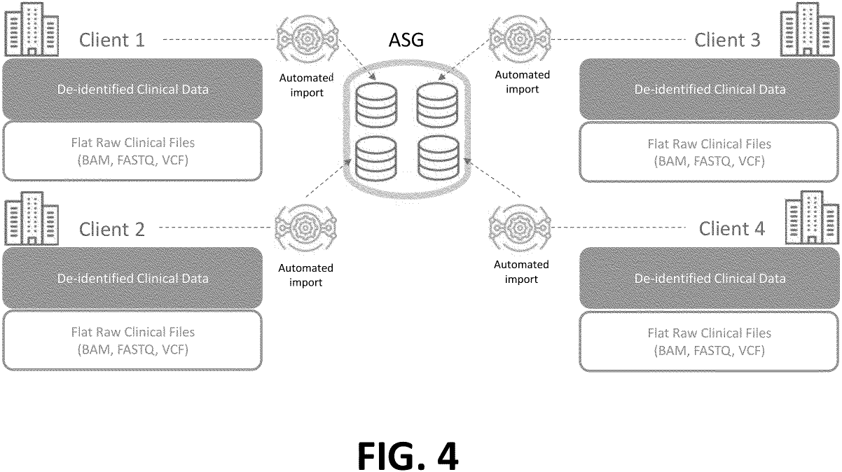

Furthermore, in embodiments, to ensure that each client’s PHI and sequencing data is protected within their own cloud-based domain, secure measures are in place to keep each client separate within their own portal stack domain (FIG. 4: The ASG Portal Infrastructure).

In embodiments, each customer portal is running on a totally unique and independent set of resources (Stack, DB, storage bucket). In embodiments, genetic and clinical files are stored in S3 bucket unique to each customer. In embodiments, the storage for each customer is encrypted with a unique set of keys. In embodiments, a unique combination of access ID / User / encryption key is created for every customer.

In embodiments, the cloud-based HIPAA compliant environment comprises software as a service (SaaS). In embodiments, the SaaS is Amazon’s HIPAA compliant cloud-storage services.

### Market Need For Genetic Testing Solution

ASG will serve in a new distribution market that is rapidly expanding as a “send-out” vertical (physicians send directly to laboratories where the test is run, and results are provided directly back to the physician). To date, there is no full, end-to-end solution that allows for initiation of a genetics program in a lab, quickly and efficiently, and at an affordable cost. ASG will offer a new solution to this need that has existed since genetic testing was launched mainstream in the early 2000′s. As indicated above, ASG offers a bioinformatics analysis pipeline for organizations that can complete a laboratory’s wet lab workflow; while formulating the entire solution to provide the ability to offer genetic testing. The partnership creates a synergistic opportunity to offer a complete technology transfer with limited overhead and risk.

The market for high throughput germline genetic testing is one of the largest growth sectors of laboratory testing in the healthcare industry. Prenatal tests including non-invasive prenatal testing (NIPT) and carrier screening account for the highest percentage of spend over the last 10 years ranging from 33 percent to 43 percent of the genetic testing market, followed by hereditary cancer tests at approximately 30 percent.

### Market Segments

Currently, genetic tests for hereditary cancer and carrier screening are run by large specialty organizations, esoteric laboratories, regional laboratories, and direct to consumer.

Additionally, laboratories that can or will internalize Next Generation Sequencing for genetic testing present an additional potential opportunity.

While the market represents a massive opportunity - it has been restricted to few companies that possess the intricate knowledge, technology, and personnel to run such testing as hospital and healthcare organizations face operational, clinical, and analytic challenges that, in most cases, cannot be overcome to launch a competitive product.

The reason for this is multifaceted, including: complexity, e.g., types of panels, number of genes offered, variants of interest, wet lab, reagents, personnel, curation, keeping up with clinical guidelines, variant reclassification, and workflows, e.g., next generation sequencing for inherited cancer and carrier screening generally requires multiple workflows (up to 6) to capture all of the variants of interest with the highest sensitivity in complex genes and regions, and may require confirmation by a secondary technology method if covered at low sequencing coverage.

The ASG assay is the only seamless technology transfer that offers carrier screening and hereditary cancer in the same product, with validation by geneticists and a full suite technology transfer.

To date, the market is dominated by large incumbent organizations which offer genetic testing as a “send-out” test directly to physicians, hospitals and large healthcare organizations. The market is divided into two segments:

Specialty laboratories: This type of laboratory offers specialized genetics testing with a focus on specific tests such as non-invasive prenatal testing (NIPT), genetic carrier screening, inherited cancer screening, exome and genome testing for rare diseases, cardiology, amongst others. Each lab specializes in a core competency and controls a large share of the US and global market. Examples include:

Large esoteric laboratories: Some larger organizations offer healthcare providers access to esoteric testing via patient service centers throughout the USA and abroad. As the market grows - these companies have begun to offer larger genetic panels to meet the needs of physicians and compete with the specialty organizations and have unparalleled access to patients and blood stations to capitalize on the market.

Direct to Consumer Genetic Testing: While this paradigm has gained a great deal of momentum in recent years, the offerings do not offer truly clinical grade testing, rather more informational.

Greater than 90% of all specialty genetic testing, which is the core product of ASG, has been offered by one of the two pathways mentioned above. While some small niche genetic tests are offered (by large sequencing companies such as Illumina and Thermo Fisher Scientific) to be internalized the platforms are not competitive and offer either only a few select genes or dated technologies. Due to the barriers of entry and complexity of development, few companies offer the assistance to healthcare organizations, regional laboratories and hospitals with the internalization of a competitive product. ASG solves this problem.

A. Vertical One: Laboratories and health systems that already have NGS Equipment:

One of the most valuable assets that ASG provides is the fact that the pipeline can be customized to suit the requirements of the end user, regardless of geography, size of lab, expertise, FTE’s, to list a few. The pipeline can also be customized (verification and validation) on a number of different sequencing machines.

i. Illumina Installed Base Opportunity:

ASG ran initial verification and validation on Illumina sequencing instruments. The reason for this being quite simple, this is our target market and these potential customers already own/lease this equipment. Additionally, they have already built out their molecular labs containing personnel and expertise to adopt ASG in a seamless manner. In embodiments, the Illumina sequencing instrument comprises Illumina NextSeq, Illumina HiSeq, or Illumina NovaSeq. The install base will already have 80%-90% of the equipment listed below in the lab with unused time and supplies that can be allocated to ASG. Every moment that an NGS sequencer is not running, it is losing money; ASG is a seamless solution to maximize already established equipment without requiring that existing labs obtain additional sequencing technology, other than the reagents and probes necessary for the reactions performed during amplificant and assaying.

ii. The non-invasive prenatal test (NIPT) opportunity:

NIPT represents the largest revenue and volume opportunity that currently exists in the genetics women’s health space. Now, with major clinical society committees (ACOG, ACMG) changing their guidelines to include all pregnancies, the standard of care for determination of fetal aneuploidy, the technology is also widely used to determine gender at as early as 9 weeks of pregnancy. Because of the lucrative nature of the test, and potential to retain patients at local and regional institutions, NIPT has been widely adopted as an LDT by multiple healthcare systems and regional laboratories, most run on the Illumina NextSeq platform. Due to the capacity to run tens of thousands of tests per month on the NextSeq, the equipment is often left idle, as it was internalized solely for this purpose. ASG presents a seamless solution to offer 2 additional lines of testing on the same equipment with a very similar workflow and requiring virtually no upfront costs of development beyond validation of the LDT.

Under this model, the Illumina NextSeq can be run 2-times per week for NIPT and 2-times per week for ASG (carrier or cancer); thus, increasing the overall output and revenue by greater than 100%. A key component of this opportunity is the volume attributed to these laboratories. All labs running NIPT internally must have a volume of at least 3,000-5,000 units per year. Most do not make the NGS leap until there are >5,000 annual units. There is a direct relationship between NIPT and carrier screening related to the patient profile, because the standard of clinical prenatal care is to prescreen for genetic carrier disease and then test for potential abnormal pregnancies.

B. Vertical Two: Laboratories with Molecular Laboratory Footprint, Medium Build-Out:

An already established molecular lab, not yet running genetics and may require limited additional capital equipment, perhaps only a sequencer.

The second vertical that ASG can penetrate has fractionally more start up requirements (i.e. limited equipment) than vertical one, however still limited barrier to entry:

Knowledge barrier: laboratories in vertical two have already committed to a molecular offering. The departments have invested in licensing, personnel, space, FTE’s, and most importantly, are already running a number of assays in with parallel start-up to ASG.

C. Vertical Three: No Established Molecular Division with a CLIA Laboratory

Laboratories and health systems that do not have an established molecular division; but have a women’s health testing laboratory that would like to penetrate the genetics market:

As discussed, the largest barrier to entry that laboratories have faced since the launch of genetics (ASG: hereditary cancer and carrier screening) has been the requirement to run multiple workflows, technologies and chemistries ensuring capture of all the necessary genes/variants of interest in a large panel.

Cost: multiple workflows require numerous capital equipment expenditures and full-time employees. This, alone, drives the price of the assay up to a point where it is not economically feasible to run the test, as each sample would lose money for the institution.

Complexity: While NGS has been around for years as a genetic testing technology, there are limited individuals that can create an assay to perform to the level of a send-out alternative.

ASG has solved the workflow challenge, eliminating the barriers to entry, allowing the entry into the market of laboratories that do not currently own an NGS sequencer, but plan to enter the space, with supporting patient volumes.

Example Case Study, Vertical Three: Over the last 5-10 years, the landscape in women’s health has changed in the USA. No longer do we see the majority of OBGYN practices independently owned. Due to overhead, liability/malpractice, and demand to see more patients as a result of declining reimbursement, practices are electing to merge or be purchased by larger organizations. Specifically, in the USA there are a number of massive “super groups” that employ thousands of OBGYNs and are responsible for millions of patients’ lives. In many circumstances, equity firms and other private investors have an ownership or equity stake -converting what was once a bottom line of clinical care to economic viability.

As a result, these groups have turned to raising laboratories to ensure that all testing remains within the four walls of the entity. As women’s health is the primary focus, there is a substantial opportunity to drive revenue through genetic testing as a revenue stream.

### Laboratory Developed Test (LDT)

Due to fact that ACMG and ACOG primarily recommend testing for 6 genes (CFTR, FMR1, HBA1, HBA2, HBB and SMN) and 5 common diseases (Cystic Fibrosis, Fragile-X Syndrome, Hemoglobinopathies, Sickle Cell and Spinal Muscular Atrophy), the larger expanded panels may not be covered by insurance in the United States and the patient must pay out-of-pocket. In lieu of these constraints, the ASG testing panels have been designed using these well-established clinical guidelines from ACMG, newborn screening guidelines from American College of Obstetrics and Gynecology, ACOG expanded carrier testing and the genomic content assessed routinely in persons of Ashkenazi Jewish descent because of the increased carrier frequency in this population. The ASG scientific team applied these principles to identify the most relevant genes/diseases to be included on the panels which are clinically actionable and have a carrier frequency higher than 1 in 500 across multiple ethnicities (i.e. Pan-ethnic panel). Additionally, the genes of interest were mapped against ClinVar to ensure all clinically relevant variants with a minimum of 2 stars were included in the panel to increase disease detection rate. Using this exercise, we have constructed a clinically-actionable and-disease relevant germline testing panel of 155 autosomal and X-linked recessive carrier genes and 90 autosomal dominant oncology genes. In embodiments, the hereditary cancer and carrier panel is built to ensure compliance with all major insurance companies.

In embodiments, the assay is for cancer or carrier screening. In embodiments, each sample is analyzed using an ASG chemistry kit. In embodiments, the sequencing kit is a novel capture kit dedicated for sequencing the genes within the scope of ASG. In embodiments, the ASG chemistry kit does not require extensive equipment and reagent use. In embodiments, the main instrumentation required is an Illumina sequencer.

The simplified chemistry workflow allows laboratories that do not have previous experience in molecular biology techniques to implement a workflow seamlessly with little-to-no overhead with a low-learning curve and limited troubleshooting. In embodiments, the chemistry is performed with limited steps compared to conventional hereditary cancer and carrier panels. In embodiments, the chemistry is performed in less time than standard NGS workflows.

In embodiments, the sequencing kit comprises designed probes for optimal capture of the gene, regions of interest. In embodiments, the probes are molecular inversion probes or padlock probes. In embodiments, the probe set screen for at least one of (SNVs), indels, (CNVs), homologous regions, and pseudogenes. In embodiments, the probe set screens for the probes screen for at least one of (SNVs), indels, (CNVs), homologous regions, and pseudogenes. In one embodiments, the probe set screens for 85 hereditary cancer genes and 155 carrier genes.

In embodiments, the assay combines the reagent components with the patient’s genomic DNA (gDNA) in a single tube process, limiting transfer steps and reducing outside contamination. In embodiments, the entire wet-lab bench work is reduced to 90 minutes of hands-on time due to the design of the chemistry and does not require multiple purification steps as observed in other chemistries to remove impurities that increase laboratory complexity and the potential for sample mixup. In embodiments, the majority of the assay runtime is made up of hands-off processes that include 16-24-hour hybridization, i.e., the binding of the gDNA to the synthetic oligonucleotides that targets the genomic regions of interest, and a ~24-hour run processing time on the sequencing instrument.

All of the systems that we are implementing as part of our development includes multiple quality control metrics to ensure that the system is transferable. Most importantly, each laboratory’s test assay performs differently across different end users and there will be some variability of the output data. To ensure that our platform can account for these variables, we are validating the assay performance with a strong baseline metrics to ensure we identify “user” error vs “assay performance” error. The overall wet-lab design of the test assay, stratifying into a simple workflow with less hands-on technologist time minimizes error rate.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A computer-implemented method for generating a report data structure for a genetic testing request that is received from an integrated client device, the computer-implemented method comprising:
contacting a sample from a subject with an oligonucleotide or primer set, said set comprising at least one oligonucleotide probe or primer pair, wherein the at least one oligonucleotide probe or primer pair is labelled and configured to bind to at least one nucleic acid sequence in the sample;
amplifying the at least one nucleic acid sequence in the sample so as to generate at least one amplification product;
sequencing the at least one amplification product using one or more next generation sequencing operations to generate library preparation product sequencing data;
transmitting the library preparation product sequencing data from the integrated client device to a genetic testing server;
identifying, based on the library preparation product sequencing data, a sequence data structure and a client identifier for the integrated client device;
storing the sequence data structure on an encrypted storage framework and in association with the client identifier;
extracting, from the sequence data structure, a) a raw sequence data object, and b) a sample data object;
generating a sample data structure comprising the raw sequence data object and the sample data object;
generating the report data structure based on the sample data structure; and
transmitting the report data structure from the genetic testing server to the integrated client device.

2. The computer-implemented method of claim 1, wherein the nucleic acid sequence comprises an exon, a splice-site, or a promoter.

3. The computer-implemented method of claim 1, wherein the raw sequence data object is a carrier testing raw sequence data object or a cancer testing raw sequence data object.

4. The computer-implemented method of claim 1, wherein the set comprises a padlock probe.

5. The computer-implemented method of claim 1, wherein the set comprises at least one oligonucleotide probe pair.

6. The computer-implemented method of claim 5, wherein the at least one oligonucleotide probe pair comprises at least one oligonucleotide probe pair selected from Table 1 or Table 2.

7. The computer-implemented method of claim 5, wherein the at least one oligonucleotide probe pair comprises at least 25% of all oligonucleotide pairs in Table 1 or Table 2.

8. The computer-implemented method of claim 5, wherein the at least one oligonucleotide probe pair comprises at least 50% of all oligonucleotide pairs in Table 1 or Table 2.

9. The computer-implemented method of claim 5, wherein the at least one oligonucleotide probe pair comprises at least 90% of all oligonucleotide pairs in Table 1 or Table 2.

10. The computer-implemented method of claim 1, further comprising, prior to generating a sample data structure, transmitting the raw sequence data object and the sample data object to a bioinformatics module of a genetic testing server.

11. A kit, comprising
i) an oligonucleotide or primer set, said set comprising at least one oligonucleotide probe or primer pair, wherein each oligonucleotide probe or primer pair is labelled and configured to amplify in an amplification reaction at least one nucleic acid sequence in a sample; and
ii) an apparatus configured to programmatically enable the analysis of library preparation product sequencing data, the apparatus comprising at least a processor, and a memory associated with the processor having computer coded instructions therein, with the computer coded instructions configured to, when executed by the processor, cause the apparatus to
a receive, from an integrated client device, am library preparation product sequencing data;
b identify, based on the library preparation product sequencing data, a sequence data structure and a client identifier for the integrated client device;
c store the sequence data structure on an encrypted storage framework and in association with the client identifier;
d extract, from the sequence data structure, a) a raw sequence data object, and b) a sample data object;
e generate a sample data structure comprising the raw sequence data object and the sample data object;
f generate a report data structure based on the sample data structure; and
g transmit the report data structure to the integrated client device.

12. The kit of claim 11, further comprising instructions for use.

13. The kit of claim 11, wherein the nucleic acid sequence comprises an exon, a splice-site, or a promoter.

14. The kit of claim 11, wherein the raw sequence data object is a carrier testing raw sequence data object or a cancer testing raw sequence data object.

15. The kit of claim 11, wherein the set comprises a padlock probe.

16. The kit of claim 11, wherein the set comprises at least one oligonucleotide probe pair.

17. The kit of claim 16, wherein the at least one oligonucleotide probe pair comprises at least one oligonucleotide probe pair selected from Table 1 or Table 2.

18. The kit of claim 16, wherein the at least one oligonucleotide probe pair comprises at least 25% of all oligonucleotide pairs in Table 1 or Table 2.

19. The kit of claim 16, wherein the at least one oligonucleotide probe pair comprises at least 50% of all oligonucleotide pairs in Table 1 or Table 2.

20. The kit of claim 16, wherein the at least one oligonucleotide probe pair comprises at least 90% of all oligonucleotide pairs in Table 1 or Table 2.
